SEQUENCE LISTING (46 sequences)

SEQ ID NO: 1 - DNA, 21 bp, synthetic construct
  source 1..21: mol_type = other DNA
ttaatttagtgcgtgatctcc

SEQ ID NO: 2 - DNA, 20 bp, synthetic construct
  source 1..20: mol_type = other DNA
agcatctgtaatggttccat

SEQ ID NO: 3 - DNA, 42 bp, synthetic construct
  source 1..42: mol_type = other DNA
tgtaaagcaagtaaagtttgaaaccctcagggtttttcggct

SEQ ID NO: 4 - DNA, 41 bp, synthetic construct
  source 1..41: mol_type = other DNA
tgtaaagcaagtaaagtttgaaaccctcagggtttttcggc

SEQ ID NO: 5 - DNA, 38 bp, synthetic construct
  source 1..38: mol_type = other DNA
tggacagctggtgctaatagaaaagtcctaggttgaag

SEQ ID NO: 6 - DNA, 37 bp, synthetic construct
  source 1..37: mol_type = other DNA
tggacagctggtgctaatagaaaagtcctaggttgaa

SEQ ID NO: 7 - DNA, 23 bp, synthetic construct
  source 1..23: mol_type = other DNA
ggcaaatctaccaatggttctaa

SEQ ID NO: 8 - DNA, 21 bp, synthetic construct
  source 1..21: mol_type = other DNA
gcagcttattatgtgggttat

SEQ ID NO: 9 - DNA, 25 bp, synthetic construct
  source 1..25: mol_type = other DNA
tgtaaagcaagtaaagtttgaaacc

SEQ ID NO: 10 - DNA, 15 bp, synthetic construct
  source 1..15: mol_type = other DNA
tggacagctggtgct

SEQ ID NO: 11 - DNA, 20 bp, synthetic construct
  source 1..20: mol_type = other DNA
ggcatccacgaaactacctt

SEQ ID NO: 12 - DNA, 19 bp, synthetic construct
  source 1..19: mol_type = other DNA
gccgatccacacggagtac

SEQ ID NO: 13 - DNA, 40 bp, synthetic construct
  source 1..40: mol_type = other DNA
tgccgccagacagcactgtgtgaagtgtgacgtggacatc

SEQ ID NO: 14 - DNA, 39 bp, synthetic construct
  source 1..39: mol_type = other DNA
ttgccgacaggatgcagaagggcgctcaggaggagcaat

SEQ ID NO: 15 - DNA, 18 bp, synthetic construct
  source 1..18: mol_type = other DNA
ggcgtacaggtctttgcg

SEQ ID NO: 16 - DNA, 18 bp, synthetic construct
  source 1..18: mol_type = other DNA
cctggcacccagcacaat

SEQ ID NO: 17 - DNA, 20 bp, synthetic construct
  source 1..20: mol_type = other DNA
tgccgccagacagcactgtg

SEQ ID NO: 18 - DNA, 21 bp, synthetic construct
  source 1..21: mol_type = other DNA
ttgccgacaggatgcagaagg

SEQ ID NO: 19 - DNA, 18 bp, synthetic construct
  source 1..18: mol_type = other DNA
tcatctctgccccctctg

SEQ ID NO: 20 - DNA, 18 bp, synthetic construct
  source 1..18: mol_type = other DNA
tcttctgggtggcagtga

SEQ ID NO: 21 - DNA, 40 bp, synthetic construct
  source 1..40: mol_type = other DNA
gtgcaggaggcattgctgatgactgatgcccccatgttcg

SEQ ID NO: 22 - DNA, 39 bp, synthetic construct
  source 1..39: mol_type = other DNA
ccaactgcttagcacccctggtggcatggactgtggtca

SEQ ID NO: 23 - DNA, 24 bp, synthetic construct
  source 1..24: mol_type = other DNA
gttgtcatacttctcatggttcac

SEQ ID NO: 24 - DNA, 23 bp, synthetic construct
  source 1..23: mol_type = other DNA
catccatgacaactttggtatcg

SEQ ID NO: 25 - DNA, 23 bp, synthetic construct
  source 1..23: mol_type = other DNA
ctcctggtgattcttcttcagga

SEQ ID NO: 26 - DNA, 23 bp, synthetic construct
  source 1..23: mol_type = other DNA
ctcctggtgattcttcttcagca

SEQ ID NO: 27 - DNA, 22 bp, synthetic construct
  source 1..22: mol_type = other DNA
cagcttattatgtgggttatgg

SEQ ID NO: 28 - DNA, 18 bp, synthetic construct
  source 1..18: mol_type = other DNA
ggtgccagggcagtgata

SEQ ID NO: 29 - DNA, 18 bp, synthetic construct
  source 1..18: mol_type = other DNA
tgccagggtacatggtgc

SEQ ID NO: 30 - DNA, 19 bp, synthetic construct
  source 1..19: mol_type = other DNA
ggtgccagggcagtgatag

SEQ ID NO: 31 - DNA, 21 bp, synthetic construct
  source 1..21: mol_type = other DNA
tgccctggcacccagcacaca

SEQ ID NO: 32 - DNA, 20 bp, synthetic construct
  source 1..20: mol_type = other DNA
tggcgtacaggtctttgctt

SEQ ID NO: 33 - DNA, 19 bp, synthetic construct
  source 1..19: mol_type = other DNA
tgccagggtacatggtgca

SEQ ID NO: 34 - DNA, 18 bp, synthetic construct
  source 1..18: mol_type = other DNA
ccaccatgtaccctgggt

SEQ ID NO: 35 - DNA, 23 bp, synthetic construct
  source 1..23: mol_type = other DNA
ctcctggtgattcttcttcaggt

SEQ ID NO: 36 - DNA, 23 bp, synthetic construct
  source 1..23: mol_type = other DNA
ctcctggtgattcttcttcagca

SEQ ID NO: 37 - DNA, 23 bp, synthetic construct
  source 1..23: mol_type = other DNA
ctcctggtgattcttcttcacga

SEQ ID NO: 38 - DNA, 23 bp, synthetic construct
  source 1..23: mol_type = other DNA
ctcctggtgattcttcttctgga

SEQ ID NO: 39 - DNA, 23 bp, synthetic construct
  source 1..23: mol_type = other DNA
ctcctggtgattcttcttaagga

SEQ ID NO: 40 - DNA, 23 bp, synthetic construct
  source 1..23: mol_type = other DNA
ctcctggtgattcttcttcagct

SEQ ID NO: 41 - DNA, 22 bp, synthetic construct
  source 1..22: mol_type = other DNA
cagcttattatgtgggttatcg

SEQ ID NO: 42 - DNA, 22 bp, synthetic construct
  source 1..22: mol_type = other DNA
cagcttattatgtgggttatct

SEQ ID NO: 43 - DNA, 22 bp, synthetic construct
  source 1..22: mol_type = other DNA
cagcttattatgtgggttaacg

SEQ ID NO: 44 - DNA, 22 bp, synthetic construct
  source 1..22: mol_type = other DNA
cagcttattatgtgggttctcg

SEQ ID NO: 45 - DNA, 22 bp, synthetic construct
  source 1..22: mol_type = other DNA
cagcttattatgtgggtaatcg

SEQ ID NO: 46 - DNA, 22 bp, synthetic construct
  source 1..22: mol_type = other DNA
cagcttattatgtgggttatgt

(19) United States
(12) Patent Application Publication
(10) Pub. No.: US 2024/0209462 A1
(43) Pub. Date: Jun. 27, 2024

(54) A METHOD FOR DETECTION OF TARGET NUCLEIC ACID USING ISOTHERMAL AMPLIFICATION

(71) Applicants: Nanyang Technological University, Singapore (SG); Agency for Science, Technology and Research, Singapore (SG)

(72) Inventors: Meng How TAN, Singapore (SG); Kean Hean OOI, Singapore (SG)

(21) Appl. No.: 18/577,860

(22) PCT Filed: Jul. 12, 2022
(86) PCT No.: PCT/SG2022/050489

§ 371 Date: Jan. 9, 2024

(30) Foreign Application Priority Data
Jul. 9, 2021 (SG) 10202107557T

(51) Int. Cl. C12Q 1/70; C12Q 1/6818; C12Q 1/6844

(52) U.S. Cl. CPC C12Q 1/701; C12Q 1/6818; C12Q 1/6844

(57) ABSTRACT

Various embodiments relate generally to the field of nucleic acid amplification and detection, in particular isothermal nucleic acid amplification and the detection of the amplicons using designated detection probes. Moreover, various embodiments also relate to methods for determining the presence or quantity of a target nucleic acid molecule in a sample using isothermal amplification. The detection probe is a single-stranded probe that recognises a probe binding site within target amplicons. The detection probe comprises at least one 3′end nucleotide mismatch and a quencher-fluorophore pair at the opposite ends of the probe. Following hybridization of the detection probe to the target amplicons, a DNA polymerase with 3′-5′ exonuclease activity can cleave the detection probe at the 3′ end nucleotide mismatch to release a 3′-terminal probe fragment comprising the quencher or fluorophore, thus generating signals.

### CROSS-REFERENCE TO RELATED APPLICATION

This application claims the benefit of priority of Singapore Patent Application No. 10202107557T filed 9 Jul. 2021, the content of which being hereby incorporated by reference in its entirety for all purposes.

### TECHNICAL FIELD

Various embodiments relate generally to the field of nucleic acid amplification and detection, in particular isothermal nucleic acid amplification and the detection of amplicons using designed detection probes. Moreover, various embodiments also relate to methods and kits for determining the presence or quantity of a target nucleic acid molecule in a sample using isothermal amplification.

### BACKGROUND

COVID-19 is a highly infectious respiratory disease caused by the SARS-CoV-2 coronavirus. A key approach to limit viral transmission is to conduct regular and extensive testing. At present, real-time quantitative polymerase chain reaction (RT-qPCR) serves as the gold standard method to detect the virus. However, it requires expensive instrumentation and specialized skills to perform, and thus can only be carried out in centralized, wellfunded facilities. Furthermore, RT-qPCR assays have a slow turnaround time, as samples must be transported from collection points to test facilities and the assay itself takes at least 1.5 h to set up and run. As an alternative to RT-qPCR, antigen rapid test (ART) has gained popularity in many countries due to its ease-of-use, fast sample-to-result time, and low cost. Nevertheless, a major drawback of ART is its poor sensitivity compared to nucleic acid amplification tests. Hence, ARTs will miss many infected individuals with low to moderate viral loads. Consequently, there is still a continuous demand for better molecular diagnostic tests that can be deployed in point-of-need situations.

Isothermal amplification methods can address the shortcomings of RT-qPCR and ART. First, they allow samples to be processed at a single temperature. As a result, simple and low-cost devices, such as a heat block or incubator, may be utilized in place of the costly thermal cyclers required for RT-qPCR. Second, when properly designed, isothermal amplification assays can exhibit a sensitivity several-fold better than that of ARTs. Currently available isothermal amplification approaches include rolling circle amplification (RCA) loop-mediated isothermal amplification (LAMP) recombinase polymerase amplification (RPA) nucleic acid sequence based amplification (NASBA) transcription-mediated amplification (TMA) helicase dependent amplification (HDA) exponential amplification reaction (EXPAR) and strand displacement amplification (SDA) among which LAMP has proven to be the most popular so far. In brief, LAMP relies on a set of four core primers (two “inner primers” termed FIP and BIP as well as two “displacement primers” termed F3 and B3), which recognize six distinct regions at the target locus. Moreover, extra primers are usually added to boost amplification efficiency. The most commonly added primer set is the “loop primers” (termed LF and LB), which are designed to anneal to the single stranded loop regions in the dumbbell structure generated during the reaction. Alternatively, two other primer sets that may be utilized include the “stem primers”, which target the single-stranded region in the center of the dumbbell structure and the “swarm primers”, which hybridize to the template strand opposite to that of FIP or BIP so as to reveal the binding sites for the inner primers.

A variety of sequence-independent methods have been applied to detect an amplified product. Such methods typically rely on (1) the turbidity caused by precipitated magnesium pyrophosphate, (2) coffee-ring formation on colloid-crystal substrates, (3) formation of DNA-magnetic bead aggregates on filter paper, (4) melting and annealing curve analysis, (5) luciferase-catalyzed bioluminescence, (6) electrochemiluminescence, (7) colorimetric dyes, (8) fluorescent dyes that bind to double-stranded DNA, or (9) agarose gel electrophoresis. Today, numerous LAMP assays for COVID-19 have been developed and commercialized based on some of these sequence-independent methods.¹⁻⁵ However, isothermal amplification often generates nonspecific products even without a template present. In particular, the large number of long primers used in LAMP results in a heightened risk of primer dimers forming. Consequently, sequence-independent detection methods are susceptible to giving false positive outcomes as they merely indicate successful amplification of DNA and do not confirm the presence of a desired target.

In contrast, sequence-specific detection methods enable the identification of bona fide amplicons and guard against spurious by-products. Furthermore, they allow for one-pot multiplexing, whereby several distinct targets are queried simultaneously in a single reaction.⁶ Over the years, multiple modes of sequence-specific detection have been developed. A few of them have also been used to detect SARS-CoV-2 recently.^{8,9-12} However, existing sequence-specific detection approaches suffer from various shortcomings that hamper their widespread adoption. First, in some methods, there is no additional probe recognizing a region of the amplicon that is separate from the primer binding sites. Hence, if the primers themselves are generating undesirable side products, such methods may give false positive results as well. Second, in some approaches, the LAMP primers are artificially extended, for example, with universal sequences. These extensions may affect amplification, for example, by interfering with primer binding or DNA polymerization. Third, a few methods require elaborate primer or probe design and therefore are not user-friendly. Fourth, for the LUX primer and HyBeacon probe, the precise mechanism of quenching and dequenching remains unclear. In addition, the fluorophores that may be utilized are restricted to those that exhibit self-quenching behaviour. Fifth, for methods that rely on base quenching, target site selection is constrained by the requirement for a specific adjacent nucleotide. Moreover, fluorescence may be affected by other nearby nucleotides as well. Sixth, for approaches that require the use of ethidium bromide, the dye is a mutagen and cannot be handled by a non-specialist. The intercalated ethidium bromide may also affect amplification efficiency. Seventh, in the LightCycler methodology, two separate probes must hybridize to adjacent nonoverlapping sequences at the target locus for fluorescence resonance energy transfer to take place. Unfortunately, this can be difficult to achieve with short amplicons. Eighth, certain methods are challenging to multiplex. For example, with LightCycler probes, one must be careful of crosstalk between donor-acceptor pairs, while for the PEI-LAMP technique, it is hard to decipher a mixture of colours in a precipitate. Ninth, for toehold switches and probes that fold back to form hairpins (such as molecular beacons), design can be challenging. The intramolecular interaction can serve as an undesirable source of competition for intermolecular target hybridization. There is also a delicate balance between hairpin stability and target hybridization. If one tries to reduce the intramolecular interaction, the hairpin may be more prone to melting under LAMP reaction conditions, leading to high background noise. Tenth, methods that depend on RNase H can only be applied on DNA targets. For RNA targets like SARS-CoV-2, the reaction must include a reverse transcription (RT) step, which use random DNA primers. The RNase H enzyme will cleave the RNA substrate once the RT primers bind.

Therefore, there is still need in the art for an alternative sequence-specific detection method to address the drawbacks of existing approaches. In particular, there is a need in the art for sequence-specific detection methods that enhance the specificity and sensitivity of existing approaches without compromising speed while also being affordable, asset-light, and simple-to-use.

### SUMMARY

In a first aspect, there is provided a method for determining the presence or quantity of a target nucleic acid molecule in a sample using isothermal amplification, the method comprising:
(a) combining an isothermal amplification reaction mixture, a DNA polymerase with 3′-5′ exonuclease activity, and a detection probe with the sample (suspected of containing the target nucleic acid molecule),
   wherein the isothermal amplification reaction mixture comprises a primer set of at least two primers, wherein each primer recognises a distinct primer binding site within the target nucleic acid molecule,
   wherein the detection probe is a single-stranded probe that recognises a probe binding site within target amplicons, said probe binding site being different from and non-overlapping with any one of the primer binding sites and
   wherein the detection probe comprises at least one 3′ end nucleotide mismatch and a quencher-fluorophore pair at opposite ends of the probe at a distance that allow the quencher to quench the fluorophore signal, wherein either the fluorophore or the quencher are attached to the 3′ end of the probe downstream of or at the site of the mismatch,
   wherein the detection probe can hybridize to said target amplicons under isothermal amplification assay conditions except for the 3′ end nucleotide mismatch and form a double-stranded probe:target complex,
(b) amplifying the target nucleic acid molecule under isothermal amplification assay conditions that allow:
   i. generation of the target amplicons;
   ii. hybridization of the detection probe to the target amplicons to form the probe:target complex; and
   iii. cleavage of the detection probe at the 3′ end nucleotide mismatch by the DNA polymerase with 3′-5′ exonuclease activity to release a 3′-terminal probe fragment comprising the quencher or fluorophore; and
(c) detecting and optionally quantifying the released probe fragments to determine the presence and optionally amount of the target nucleic acid molecule in the sample.

In various embodiments, the DNA polymerase with 3′-5′ exonuclease activity is a high-fidelity DNA polymerase.

In various embodiments, the isothermal amplification is loop-mediated isothermal amplification (LAMP), and the primer set comprises at least 4 or 6 primers comprising two inner primers (FIP and BIP) and two outer primers (F3 and B3), and optionally two loop primers (LF and LB).

In various embodiments, the probe binding site lies between the binding sites of the inner primers.

In various embodiments, the primer set further comprises two swarm primers.

In various embodiments, the at least one 3′ end nucleotide mismatch comprises a single 3′ end nucleotide mismatch.

In various embodiments, the single 3′ end nucleotide mismatch is positioned at the last or second to last nucleotide relative to the 3′ end of the detection probe.

In various embodiments, the at least one 3′ end nucleotide mismatch comprises two 3′ end nucleotide mismatches.

In various embodiments, the two 3′ end nucleotide mismatches are the last two nucleotides relative to the 3′ end of the detection probe.

In various embodiments, the detection probe is 17-30 nucleotide bases in length.

In various embodiments, the quencher is attached to the 5′ end of the detection probe and the fluorophore is attached to the 3′ end of the detection.

In various embodiments, the quencher is a double quencher.

In various embodiments, the detection method in step (c) is lateral flow detection or fluorescence detection.

In various embodiments, the method is a multiplexing method and is for determining the presence, absence and optionally amount of two or more target nucleic acid molecules in the sample, wherein the method uses one or more primer sets and/or one or more detection probes for each target nucleic acid molecule or for multiple related target nucleic acid molecules.

In various embodiments, the target nucleic acid molecule is a nucleic acid of a pathogen, optionally a human pathogen, preferably a bacterial, fungal, parasite or viral nucleic acid molecule or a cDNA reverse transcript of a bacterial, fungal, parasite or viral RNA.

In various embodiments, the target nucleic acid molecule is a nucleic acid of a coronavirus, influenza virus, paramyxovirus or enterovirus.

In various embodiments, the target nucleic acid molecule is a nucleic acid of SARS-CoV-2 virus.

In various embodiments, the sample has not been subjected to any nucleic acid purification or extraction step prior to step (a) of the method.

In various embodiments, step (a) further comprises pyrophosphatase.

In another aspect, there is provided a use of the detection probe as defined herein for determining the presence or quantity of a target nucleic acid molecule in a sample using an isothermal amplification method.

In another aspect, there is provided a kit for determining the presence or quantity of a target nucleic acid molecule in a sample using isothermal amplification, the kit comprising: an isothermal amplification reaction mixture;
a DNA polymerase with 3′-5′ exonuclease activity; and
a detection probe,
wherein the isothermal amplification reaction mixture comprises a primer set of at least two primers, wherein each primer recognizes a distinct primer binding site within the target nucleic acid molecule,
wherein the detection probe is a single-stranded probe that recognizes a probe binding site within target amplicons, said probe binding site being different from and non-overlapping with any one of the primer binding sites,
wherein the detection probe comprises at least one 3′ end nucleotide mismatch and a quencher-fluorophore pair at opposite ends of the probe at a distance that allow the quencher to quench the fluorophore signal, wherein either the fluorophore or the quencher are attached to the 3′ end of the probe downstream of or at the site of the mismatch, wherein the detection probe can hybridize to said target amplicons under isothermal amplification assay conditions except for the 3′ end nucleotide mismatch and form a double-stranded probe:target complex.

In various embodiments, the kit further comprises pyrophosphatase.

### Definitions

The following words and terms used herein shall have the meaning indicated:

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

Unless otherwise defined, all technical and scientific terms used herein have the same meaning as commonly understood by one of ordinary skill in the art. The singular terms “a,” “an,” and “the” include plural referents unless context clearly indicates otherwise. Similarly, the word “or” is intended to include “and” unless the context clearly indicates otherwise. The term “comprises” means “includes.” In case of conflict, the present specification, including explanations of terms, will prevail.

As used herein, the term “LANTERN” refers to “Luminescence from Anticipated Target due to Exonuclease Removal of Nucleotide mismatch” and is a descriptive acronym of the method according to various embodiments described herein developed by the inventors of the application. Accordingly, the term “LANTERN assay” may be used herein to refer to the method according to various embodiments described herein. Moreover, the term “LANTERN probe” refers to the detection probe according to various embodiments described herein used in said method and developed by the inventors of the application.

As used herein, the term “at least one”, as used herein, means one or more, for example 2, 3, 4, 5, 6, 7, 8, 9 or more. If used in relation to a component or agent, the term does not relate to the total number of molecules of the respective component or agent but rather to the number of different species of said component or agent that fall within the definition of broader term.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the term “about”, in the context of concentrations or amounts of components, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

### BRIEF DESCRIPTION OF THE DRAWINGS

Various embodiments will be better understood with reference to the detailed description when considered in conjunction with the non-limiting examples and the accompanying drawings.

FIG. 1 illustrates an overview of the method according to various embodiments described herein. (A) Schematic diagram depicting a 3′ mismatched single-stranded DNA (ssDNA) probe that has a quencher and fluorophore attached at opposite ends. The 3′ mismatched nucleotide will be cleaved off by the DNA polymerase after hybridization of the probe to the target amplicon, thereby separating the fluorophore and quencher, which then results in a fluorescence signal; and (B) Schematic diagram depicting the positions of the FAM fluorophore (F) and the quenchers (Q) on a probe designed to target the stem region of the SARS-CoV-2 S-gene amplicon

FIG. 2 shows the development and characterization of a prototype LANTERN assay for COVID-19: (a) Fluorescence measurements after 25 minutes of RT-LAMP, whereby 2E4 copies of purified synthetic SARS-CoV-2 RNA template were added into each reaction together with 2 μM of either single quenched or double quenched LANTERN probes against the viral amplicon. Data represent mean±s.e.m. (n=3 [double quencher] or 4 [single quencher] biological replicates). (*** P<0.001, n.s.: not significant; two-sided Student's t-test). In each column pair under single or double quencher, 2E4 RNA is the column bar on the left and NTC is the column bar on the right of the 2E4 RNA bar; (b) Evaluating the effect of 0.5 U pyrophosphatase (PPase) on the LANTERN assay. The fluorescence measurements were taken after 25 minutes of RT-LAMP, whereby 2E4 copies of synthetic SARS-CoV-2 RNA template in heat-inactivated saliva were added into each reaction together with variable amounts (0.5, 1, or 2 μM) of double quenched probes against the viral amplicon. LAMP primer sets designed to amplify both the S-gene of SARS-CoV-2 and human GAPDH were used to simulate a situation whereby simultaneous amplification of a human internal control with the S-gene may disrupt the fluorescence signal indicating presence of the virus. Data represent mean±s.e.m. (n=2 biological replicates). (* P<0.05, ** P<0.01, n.s.: not significant; two-sided Student's t-test). In each amount column pairing, 2E4 RNA is the column bar on the left and NTC is the column bar on the right of the 2E4 RNA bar; (c) Optimizing the concentrations of PPase and Q5 high-fidelity DNA polymerase. The fluorescence measurements were taken after 25 minutes of RT-LAMP, whereby 2E4 copies of purified synthetic SARS-CoV-2 RNA template were added into each reaction together with either 0.5 or 1 μM of double quenched probes against the viral amplicon. Data represent mean±s.e.m. (n=3 biological replicates); (d, e) End point visualization of sample tubes with a gel illuminator after 25 minutes of RT-LAMP. Different copies of purified synthetic SARS-CoV-2 RNA were tested and 0.5 μM of double quenched probes against the viral amplicon were used. Either d 0.5 U PPase and 0.5 U Q5 or e 0.8 U PPase and 0.8 U Q5 were added in each reaction; (f) Analytical LoD for purified synthetic SARS-CoV-2 RNA. Fluorescence measurements here were taken after 25 minutes of RT-LAMP with 0.5 μM of double quenched probes against the viral amplicon. Data represent mean±s.e.m. (n=4 biological replicates); (g) Analytical LoD for synthetic SARS-CoV-2 RNA in heat-inactivated saliva. Fluorescence measurements here were taken after 25 minutes of RT-LAMP with 0.5 μM of double quenched probes against the viral amplicon. Data represent mean±s.e.m. (n=3 biological replicates); and (h) Evaluating cross-reactivity with human nucleic acids. Fluorescence measurements here were taken after 25 minutes of RT-LAMP, whereby various viral or human templates were used as input. Data represent mean±s.e.m. (n=3 biological replicates).

FIG. 3 shows time courses of the fluorescence intensity in the LANTERN assay. 2E4 copies of purified synthetic SARS-CoV-2 RNA were added into each reaction together with 2 μM of either single quenched or double quenched probes against the viral amplicon. Fluorescence was monitored in a real-time PCR machine, with readings taken every minute. Data represent mean±s.e.m. (n=3 [double quencher] or 4 [single quencher] biological replicates).

FIG. 4 shows the effect of pyrophosphatase (PPase) on the LANTERN assay. 2E4 copies of synthetic SARS-CoV-2 RNA template in heat-inactivated saliva were added into each reaction together with variable amounts (0.5, 1, or 2 μM) of double quenched probes against the viral amplicon. S-gene LAMP primers and human GAPDH LAMP primers were used together to mimic a situation whereby concurrent amplification of a human internal control with the S-gene may interfere with the fluorescence signal indicating presence of the virus. Fluorescence was monitored in a real-time PCR instrument, with measurements taken every minute. Data represent mean±s.e.m. (n=3 biological replicates).

FIG. 5 shows the optimization of the concentrations of PPase and high-fidelity DNA polymerase. 2E4 copies of purified synthetic SARS-CoV-2 RNA template were added into each reaction together with 1 μM (left panel) or 0.5 μM (right panel) of double quenched probes against the viral amplicon. Fluorescence was monitored in a real-time PCR instrument, with measurements taken every minute. Overall, a greater amount of Q5 polymerase resulted in faster reaction kinetics. Data represent mean±s.e.m. (n=3 biological replicates). Each line and combined concentrations of the PPase and Q5 have been designated a number from 1-7 for ease of reference. At the 40 minute mark in the graph for 1 μM and 0.5 μM, the order of the lines from highest RFU to lowest are as follows: (1 μM)=6>5>4>3>2>1>7; (0.5 μM)=4>6>3>2>5>1>7.

FIG. 6 shows the analytical sensitivity of the LANTERN assay with 0.5 μM of double quenched probes against the viral amplicon: (a) Graph of time courses of the fluorescence intensity measured every minute using a real-time PCR instrument for purified synthetic SARS-CoV-2 RNA. Data represent mean±s.e.m. (n=4 biological replicates); and (b) Graph of time courses of the fluorescence intensity measured every minute using a real-time PCR instrument for synthetic SARS-CoV-2 RNA in heat-inactivated donor saliva. Data represent mean±s.e.m. (n=3 biological replicates). At the 40 minute mark in graphs of (a) and (b), the order of the lines from highest RFU to lowest are as follows: (a)=2E4>2E3>2E2>2E1>2>NTC; (b)=2E4 and 2E3>2E2 and 2E1>2>NTC.

FIG. 7 shows the cross-reactivity with human RNA or DNA; (a) Graph of time courses of the fluorescence intensity measured using a real-time PCR instrument for various viral (2E1 or 2E4 copies per reaction) or human (10 ng per reaction) templates. 0.5 μM of double quenched probes against the SARS-CoV-2 amplicon were used in each reaction. Data represent mean±s.e.m. (n=3 biological replicates); and (b) End point visualization of sample tubes with a gel illuminator after 25 minutes of RT-LAMP. The two biological replicates shown here are distinct from those obtained using the real-time PCR instrument shown in (a).

FIG. 8 shows the incorporation of a human internal control into the LANTERN assay: (a) Evaluation of two different Cy5-conjugated ACTB (beta actin) probes in the presence or absence of swarm primers. The concentrations used were 0.5 μM stem probe, 0.5 μM loopB probe, or 0.25 μM stem probe and 0.25 μM loopB probe. Fluorescence measurements here were taken after 25 minutes of RT-LAMP. Data represent mean±s.e.m. (n=3 [without swarm] or 4 [with swarm] biological replicates). (** P<0.01, *** P<0.001, n.s.: not significant; one-sided Student's t-test). In each of the stem, LoopB and Stem+LoopB sections the order of the bar columns from left to right are Saliva>NTC>Saliva(+swarm)>NTC(+swarm); (b) Evaluation of a one-pot reaction with viral S-gene and human ACTB primers and probes. The template used was 2E4 copies of synthetic SARS-CoV-2 RNA alone, heat-inactivated saliva alone, or both together. Here, fluorescence was measured after 25 minutes of RT-LAMP. Data represent mean±s.e.m. (n=4 biological replicates). (^{#}P<0.1, * P<0.05; one-sided Student's t-test); (c) Strip chart showing the effect of reducing the amount of ACTB primers. RT-LAMP was performed at 65° C. with variable copies of synthetic viral RNA template in heat-inactivated saliva. The black horizontal bars among the data points in the strip chart represent the mean (n=2 [1×], 3 [0.3×], or 4 [0.5×] biological replicates).

FIG. 9 shows graphs of testing LANTERN probes and swarm primers for human ACTB. The concentrations used were 0.5 μM stem probe (left panel), 0.5 μM loopB probe (middle panel), or 0.25 μM stem probe and 0.25 μM loopB probe (right panel). Heat-inactivated donor saliva served as the template. Fluorescence was monitored over 40 minutes using a real-time PCR instrument. Data represent mean±s.e.m. (n=3 [without swarm] or 4 [with swarm] biological replicates).

FIG. 10 shows graphs of the preliminary evaluation of a one-pot reaction that contained LAMP primers and probes for the viral S-gene and human ACTB together. The template was either 2E4 copies of synthetic SARS-CoV-2 RNA alone, heat-inactivated saliva alone, or viral RNA spiked into saliva. Fluorescence intensity was monitored over 40 minutes using a real-time PCR instrument (FAM: viral target, Cy5: human internal control). Data represent mean±s.e.m. (n=4 biological replicates).

FIG. 11 shows multiplexed detection of the S-gene of SARS-CoV-2 and human ACTB. Different amounts of ACTB LAMP primers were tested. Fluorescence intensity was monitored over 40 minutes using a real-time PCR instrument (FAM: viral target, Cy5: human internal control). Data represent mean±s.e.m. (n=2 [1×], 3 [0.3×], or 4 [0.5×] biological replicates). At the 40 minute mark in graphs of (1× primers), (0.5× primers) and (0.3× primers) for each of FAM and Cy5, the order of the lines from highest RFU to lowest are as follows: FAM (1× primers)=2E4>2>2E3>2E1>2E2>NTC; (0.5× primers)=2E4>2E3>2E1>2>2E2>NTC; (0.3× primers)=2E4 and 2E1>2E2>2E3>2>NTC; and Cy5 (1× primers)=2E3>NTC>2E2>2E1>2>2E4; (0.5× primers)=NTC>2E2>2E3>2 and 2E1>2E4>NTC; (0.3× primers)=NTC>2>2E2>2E3>2E1>2E4.

FIG. 12 shows the evaluation of probes with variable mismatches: (a) Sequences of synthetic viral RNA templates (stem region) tested. Mismatches with the probe are indicated by bold red letters. The stem-targeting probe contains a mismatch at its 3′ end against the wild type (WT) sequence, so the MM1 template is actually wild type in reality; (b) Evaluation of mismatch position for a probe targeting the stem region of the S-gene amplicon. The positions were counted from the 3′ end of the probe. Fluorescence measurements here were taken after 25 min of RT-LAMP. Data represent mean±s.e.m. (n=5 biological replicates). P-values were calculated using one-sided Student's t test; (c) Comparison of a stem-targeting double mismatched probe (MM1+2) with two different single mismatched probes (MM1 and MM2). Here, fluorescence was measured after 25 min of RT-LAMP. Data represent mean±s.e.m. (n=4 biological replicates). P-values were calculated using one-sided Student's t test; (d) Sequences of synthetic viral RNA templates (loop region) tested. Mismatches with the probe are indicated by bold red letters. The loop-targeting probe contains a mismatch at its 3′ end against the wild type (WT) sequence, so the MM1 template is actually wild type in reality; (e) Evaluation of mismatch position for a probe targeting the loop region of the S-gene amplicon. Fluorescence measurements here were taken after 25 min of RT-LAMP. Data represent mean±s.e.m. (n=4 biological replicates). P-values were calculated using one-sided Student's t test. Results obtained for the loop targeting probe showed a similar trend with those obtained for the stem-targeting probe; (f) Comparison of a loop-targeting double mismatched probe (MM1+2) with two different single mismatched probes (MM1 and MM2). Here, fluorescence was measured after 25 min of RT-LAMP. Data represent mean±s.e.m. (n=2 biological replicates). P-values were calculated using one-sided Student's t test.

FIG. 13 shows how varying the position of mismatch between a probe that targeted the S-gene amplicon's stem region and its substrate affected the fluorescence signal: (a) Graph of time courses of the fluorescence intensity measured using a real-time PCR instrument for different synthetic templates. The mismatch position was counted from the 3′ end of the probe. Data represent mean±s.e.m. (n=5 biological replicates); (b) Graph comparing double mismatched probe (MM1+2) with single mismatched probes (MM1 and MM2). Fluorescence was monitored over 40 minutes in a real-time PCR instrument at 65° C. Data represent mean±s.e.m. (n=4 biological replicates).

FIG. 14 shows how varying the position of mismatch between a probe that targeted the S-gene amplicon's loop region and its substrate affected the fluorescence signal: (a) Graph of time courses of the fluorescence intensity measured using a real-time PCR instrument for different synthetic templates. The mismatch position was counted from the 3′ end of the probe. Data represent mean±s.e.m. (n=4 biological replicates); (b) Graph comparing double mismatched probe (MM1+2) with single mismatched probes (MM1 and MM2). Fluorescence was monitored over 40 minutes in a real-time PCR instrument at 65° C. Data represent mean±s.e.m. (n=2 biological replicates).

FIG. 15 show the sensitivity and specificity of detection with double mismatched probes: (a) Evaluation of different proofreading enzymes to cleave the mismatched probes, in combination with the Bst 2.0 WarmStart DNA Polymerase for the RT-LAMP reaction. Fluorescence measurements here were taken after 25 min of RT-LAMP. Data represent mean±s.e.m. (n=3 [iProof, HotStar, and Pfu], 4 [KOD], or 5 [Q5 and SuperFi] biological replicates). P-values were calculated using two-sided Student's t test; (b) Evaluation of the top two proofreading enzymes (Q5 and SuperFi) in combination with an alternative Bsm DNA Polymerase for the RT-LAMP reaction. Both Bst and Bsm polymerases have strong strand displacement activity. Fluorescence measurements here were taken after 25 min of RT-LAMP. Data represent mean±s.e.m. (n=3 biological replicates). P values were calculated using two-sided Student's t test; (c) Analytical LoD based on the original double quenched FAM-conjugated probe targeting the stem region of the S-gene amplicon and an artificial RNA template with two mismatches against the probe. Fluorescence measurements here were taken after 25 min of RT-LAMP. Data represent mean±s.e.m. (n=3 biological replicates); (d) Evaluating different human ACTB probes, each of which contained two 3′ end mismatches against the reference sequence. Fluorescence measurements here were taken after 25 min of RTLAMP. Data represent mean±s.e.m. (n=3 biological replicates). (e) Analytical LoD for a new viral S-gene probe with two 3′ end mismatches against the actual wild type target sequence. The human ACTB loopB-hybridization probe (MM1+2) was also added in the same reaction. As template for the one-pot amplification reaction, various copies of synthetic SARS-CoV-2 RNA were spiked into 0.25 ng total human RNA isolated from HEK293FT cells. To simultaneously detect the virus and the internal control, fluorescence shown here was measured in both the FAM and Cy5 channels after 25 min of RT-LAMP. Data represent mean±s.e.m. (n=4 biological replicates); (f) Similar to (e), except that the 0.25 ng total human RNA was isolated from PC9 cells. Data represent mean±s.e.m. (n=3 biological replicates); (g) Evaluating the specificity of the LANTERN assay. As the template of each reaction, 1×106 copies of synthetic RNA from a particular respiratory virus were spiked into 0.25 ng total human RNA from PC9 cells. Fluorescence was monitored in a real-time PCR instrument over 40 min. Data represent mean±s.e.m. (n=6 [COVID-19 and PavaInfluenza 4] or 3 [all others] biological replicates).

FIG. 16 shows different DNA polymerases for the LANTERN assay: (a) Graph of time courses of the fluorescence intensity measured using a real-time PCR instrument for various proofreading enzymes with 3′→5′ exonuclease activity to cleave mismatched probes. Here, the Bst 2.0 WarmStart DNA Polymerase was used for the RT-LAMP reaction. Data represent mean±s.e.m. (n=3 [iProof, HotStar, and Pfu], 4 [KOD], or 5 [Q5 and SuperFi] biological replicates): (b) Graph of time courses of the fluorescence intensity measured using a real-time PCR instrument for the top two proofreading enzymes (Q5 and SuperFi). Here, an alternative Bsm DNA Polymerase was used for the RT-LAMP reaction. Data represent mean±s.e.m. (n=3 biological replicates).

FIG. 17 shows a graph of the LoD based on the original double quenched FAM-conjugated probe targeting the stem region of the S-gene amplicon and an artificial RNA template with two mismatches against the probe. Different copies of the RNA template were tested. Fluorescence was monitored over 40 minutes in a real-time PCR instrument at 65° C. Data represent mean±s.e.m. (n=3 biological replicates).

FIG. 18 shows the evaluation of different human ACTB probes with two mismatches. Fluorescence was monitored over 40 minutes in a real-time PCR instrument at 65° C. Data represent mean±s.e.m. (n=3 biological replicates).

FIG. 19 shows the evaluation of assay sensitivity with contrived RNA specimens: (a) Analytical LoD based on a new viral S-gene probe with two 3′ end mismatches against the wild type target sequence. The reaction mix also contained the human control double mismatched (MM1+2) probe against the loopB region of the ACTB amplicon. Variable copies of synthetic SARS-CoV-2 RNA were spiked into 0.25 ng total human RNA isolated from HEK293FT cells. Fluorescence was monitored over 40 minutes in a real-time PCR instrument at 65° C. Data represent mean±s.e.m. (n=4 biological replicates); (b) Similar to (a), except that the 0.25 ng total human RNA was isolated from PC9 cells. Data represent mean±s.e.m. (n=3 biological replicates). At the 40 minute mark in the left hand graph in (a) and (b), the order of the lines from highest RFU to lowest are as follows: (a)=2E3>2E2>2E1>2E4>2>NTC; (b)=2E4>2E3>2E2>2E1>2>NTC.

FIG. 20 shows a schematic diagram of the paper craft design of a lightbox. The main casing is shown on the left side, with dotted lines indicating where the cardboard should be folded. The four independent bold lines indicate slits, while the two rectangles with diagonal lines indicate windows that should be cut out. Besides the main casing, a tube holder is shown on the right side. The two dotted circles are to be cut out for placement of sample tubes. Although the current design is for two tubes, the DIY (do-it-yourself) lightbox can be customized to contain any number of sample tubes.

FIG. 21 shows the evaluation of the LANTERN assay on direct swab or saliva samples: (a) Workflow of a COVID-19 diagnostic test for clinical samples without RNA extraction. Each NP swab or saliva sample is treated with proteinase K and heated at 95° C. for 5 min before being transferred into an RT-LAMP reaction mix containing LANTERN probes. The sample tube is then incubated at 65° C. for 30 min before detection signals are measured. The fluorescence can also be continuously monitored over time in a real-time PCR machine; (b) Analytical LoD for NP swab samples spiked with various amounts of SARS-CoV-2 produced in Vero E6 cells. 2 U of Q5 DNA polymerase was used in a 25 μL reaction volume without additional EDTA. To simultaneously detect the virus and the internal control, fluorescence shown here was measured in both the FAM and Cy5 channels after 30 min of RT-LAMP. ACTB primers were loaded at 0.3×, while S-gene primers were loaded at 1×. Data represent mean±s.e.m. (n=4 biological replicates); (c) Similar to (b), except that 2 U of Q5 DNA polymerase was used in a 25 μL reaction volume with additional 50 mM EDTA. Data represent mean±s.e.m. (n=6 biological replicates); (d) Analytical LoD for saliva samples spiked with various amounts of SARSCoV-2 produced in Vero E6 cells. 0.5 U of Q5 DNA polymerase was used in a 25 μL reaction volume without additional EDTA. Fluorescence measurements here were taken after 30 min of RT-LAMP. Data represent mean±s.e.m. (n=3 biological replicates); (e) Similar to (d), except that 1 U of Q5 DNA polymerase was used in a 50 μL reaction volume. Data represent mean±s.e.m. (n=3 biological replicates); (f) Analytical LoD for saliva samples collected in a commercially available ZeroPrep Saliva Buffer with various amounts of the coronavirus spiked in. The samples were heated at 95° C. for 5 min before being added to the reaction tubes. 1 U of Q5 DNA polymerase was used in a 50 μL reaction volume without additional EDTA. Fluorescence measurements here were taken after 30 min of RT-LAMP. Data represent mean±s.e.m. (n=5 biological replicates).

FIG. 22 shows the preliminary testing of contrived NP swab samples using original reaction conditions. Variable copies of SARS-CoV-2 produced in Vero E6 cells were spiked into clinically negative UTM. Each sample was treated with proteinase K and heated at 95° C. for 5 minutes before being added to the RT-LAMP reaction mix, which contained 0.5 U Q5 High-Fidelity DNA Polymerase. The human ACTB control was inconsistently detected in both (a) the first replicate and (b) the second replicate.

FIG. 23 shows the effect of variable amounts of Q5 enzyme on the detection of ACTB in clinically negative UTM. Each of the RT-LAMP reaction mixes contained 0.5 U PPase: (a) As a positive control, a generic DNA-binding fluorescent dye was used to detect the LAMP amplicons instead of the LANTERN probe for ACTB. The graph shows amplification curves for 8 biological replicates. All the attempts were successful in detecting the target; (b) Here, each reaction contained 0.5 μM LANTERN probe for human ACTB and 0.5 U Q5 DNA polymerase. The graph shows amplification curves for 8 biological replicates. Only 1 of the attempts was successful in detecting the target; (c) Here, each reaction contained 0.5 μM LANTERN probe for human ACTB and 1 U Q5 DNA polymerase. The graph shows amplification curves for 8 biological replicates. ACTB was successfully amplified in all the attempts, but the signal was lower for 3 of the replicates: (d) Here, each reaction contained 0.5 μM LANTERN probe for human ACTB and 2 U Q5 DNA polymerase. The graph shows amplification curves for 15 biological replicates. ACTB was reliably detected in all the attempts: (e) As a negative control, 0.5 μM LANTERN probe was tested for human ACTB and 2 U Q5 DNA polymerase on clean UTM. The graph shows amplification curves for 3 biological replicates. No fluorescence signal was observed in all the attempts.

FIG. 24 shows the analytical LoD for contrived NP swab samples spiked with variable amounts of SARS-CoV-2 produced in Vero E6 cells: (a) 2 U of Q5 DNA polymerase was used in a 25 μl reaction volume without additional EDTA. Fluorescence intensity was monitored over 40 minutes in both the FAM and Cy5 channels using a real-time PCR machine at 65° C. ACTB primers were loaded at 0.3×, while S-gene primers were loaded at 1×. Data represent mean±s.e.m. (n=4 biological replicates); (b) Similar to (a), except that 2 U of Q5 DNA polymerase was used in a 25 μl reaction volume with additional 50 mM EDTA. Data represent mean±s.e.m. (n=6 biological replicates). At the 40 minute mark in the left and right hand graph in (a) and (b), the order of the lines from highest RFU to lowest are as follows: (a) left hand graph=100>200>2000>50 and 20>2>NTC; right hand graph=NTC>50>20 and 2>200>100>2000; (b) left hand graph=200>2000>100>50>20>2>NTC; right hand graph=NTC>2>20>50>100 and 200>2000.

FIG. 25 shows the analytical LoD for contrived human saliva samples spiked with variable amounts of SARS-CoV-2 produced in Vero E6 cells. The contrived specimens were first treated with proteinase K and then heated at 95° C. for 5 minutes before being used. Two different RT-LAMP reaction volumes were tested: (a) 25 μl (containing 0.5 U of Q5 polymerase); and (b) 50 μl (containing 1 U of Q5 polymerase). Fluorescence was monitored over 40 minutes in a real-time PCR instrument at 65° C., with measurements taken every minute in the FAM and Cy5 channels. Data represent mean±s.e.m. (n=3 biological replicates). At the 40 minute mark in the left and right hand graph in (a) and (b), the order of the lines from highest RFU to lowest are as follows: (a) Left hand=20000>2000>200>100 and 50>20>>2>NTC; Right hand=NTC>20>100 and 50>2>200>2000>20000; (b) Left hand=20000>2000 and 50>100>20>2>NTC; Right hand=2>20, 50, 100, 200>NTC>2000>20000.

FIG. 26 shows the analytical LoD for saliva samples collected in a commercially available ZeroPrep Saliva Buffer with various amounts of SARS-CoV-2 spiked in. Each contrived specimen was heated at 95° C. for 5 minutes to inactivate the proteinase K in the buffer before being added to the RT-LAMP reaction mix. 1 U of Q5 DNA polymerase was used in a 50 μl reaction volume. Fluorescence was monitored over 40 minutes in a real-time PCR instrument at 65° C., with measurements taken at one-minute intervals in the FAM and Cy5 channels. Data represent mean±s.e.m. (n=5 biological replicates). At the 40 minute mark in the left and right hand graph, the order of the lines from highest RFU to lowest are as follows: Left hand=20000>2000, 200, 100>50>20>2>NTC; Right hand=2>NTC>20>50>2000>100, 200>20000.

FIG. 27 shows the evaluation of the LANTERN assay on clinical RNA samples: (a) Independent assessment of the COVID-19 diagnostic test. A total of 74 residual RNA samples were used in the evaluation, although one sample returned an invalid result since its fluorescence signals in both the FAM and Cy5 channels were below threshold levels. In the RT-qPCR experiments, a Ct value of 35.5 (based on N-gene) was estimated to be equivalent to 4 copies of the virus. 6 clinical samples gave Ct values between 37.5 and 40.0, which would correspond to less than 1 copy per reaction when extrapolated from the standard calibration curve; (b) Strip chart summarizing the results from the evaluation of a diagnostic test using clinical RNA samples. “Yes” indicates that the samples emerged positive in the assay, while “No” indicates that the samples emerged negative.

FIG. 28 shows an independent assessment of the LANTERN diagnostic assay using residual RNA samples that had previously been analysed by RT-qPCR; (a) 52 COVID-19 positive samples, with a wide range of Ct values (from 15-40), were analysed. Fluorescence intensity was monitored in both the FAM (for virus) and Cy5 (for human internal control) channels over 40 minutes. The dotted lines indicate samples that were positive in the earlier RT-qPCR analysis but turned out negative in the assay. Nevertheless, all these samples also had low viral loads, as their Ct values were at least 34.5. Since a Ct value of 35.5 in the RT-qPCR experiments was estimated to be equivalent to 4 copies of the virus, the LANTERN assay exhibits a clinical sensitivity of around 8 copies per reaction; (b) 22 COVID-19 negative samples, with undetermined Ct values, were analysed. One sample (denoted in yellow) returned an invalid result since its fluorescence signals in both the FAM and Cy5 channels were below threshold levels (represented by horizontal dashed dark green or pink lines). In each panel, the sole dotted curve indicates a false positive sample that amplified in the LANTERN assay. Hence, the diagnostic test has a specificity of 95%.

### DETAILED DESCRIPTION

The following detailed description refers to, by way of illustration, specific details and embodiments in which the invention may be practiced. These embodiments are described in sufficient detail to enable those skilled in the art to practice the invention. Other embodiments may be utilized and structural and logical changes may be made without departing from the scope of the invention. Embodiments described below in context of the detection probe are analogously valid for the respective methods and kits, and vice versa. The various embodiments are not necessarily mutually exclusive, as some embodiments can be combined with one or more other embodiments to form new embodiments.

To address the limitations of existing approaches to nucleic acid molecule detection, there is provided an alternative sequence-specific detection method as described herein that is able to achieve rapid and sensitive detection of a desired target. The sequence-specific detection method described herein is based, in part, on the proofreading capability of DNA polymerases and a specifically developed detection probe.

In particular, the method according to various embodiments described herein may be used for determining the presence or quantity of a target nucleic acid molecule in a broad range of samples using an isothermal amplification assay and a specifically developed detection probe.

As used herein, the “sample” may be any suitable sample selected from but not limited to environmental samples (e.g., soil samples, dirt samples, garbage samples, sewage samples, industrial effluent samples, air samples, water samples from a variety of water bodies such as lakes, rivers, ponds etc), food samples (e.g. samples from food intended for human or animal consumption such as processed foods, raw food material, produce, legumes, meats, fish, seafood, nuts, beverages, drinks, fermentation broths, and/or a selectively enriched food matrix comprising any of the above-listed foods, infant formulas, infant food, etc.), or biological samples. A biological sample may refer to a sample obtained from a subject that may be any eukaryotic or prokaryotic source and may be, for instance, in the form of a single cell, in the form of a tissue, or in the form of a fluid. In various embodiments, the biological sample may be biological fluids, including blood, plasma, serum, saliva and the like. In various embodiments, the biological sample may be derived from a subject, suffering from or suspected of suffering from a disease, for example an infectious disease, the subject preferably being a mammal, for example a human. Alternatively, the subject may also be an animal or plant. In various embodiments, the subject may be a human. If the method is used for pathogen detection, any sample type useful and known for such purpose may be used.

In various embodiments, the sample may not be subjected to any nucleic acid purification or extraction step prior to use in the methods described herein.

In various embodiments, the sample may be subjected to heat-inactivation in order to obtain a crude extract of the target nucleic acid molecule prior to use in the methods described herein. In various embodiments, the sample may be heated at about 95° C. for about 5 minutes alone prior to use in the methods described herein. In various embodiments, the sample may be treated with proteinase K at room temperature for 1 minute and then heated at about 95° C. for about 5 minutes. For example, the heat and proteinase K treatment may help to release the target nucleic acid molecule from inside a viral particle contained within said sample.

As used herein, the term “target” refers to the target nucleic acid to be detected but further encompasses the amplicons produced by the isothermal amplification reaction that include sequences of the target that are recognized by the primers and/or the detection probes. Accordingly, when reference is made to a target that is bound by primers or detection probes, this term typically relates to the amplicons as produced in the isothermal amplification reaction, as these are more prevalent than the original target nucleic acid. “Amplicons”, as used herein, relate to the amplified products generated starting from the template, i.e. the original target nucleic acid.

In various embodiments, the target nucleic acid molecule may be a nucleic acid sequence on a single strand of nucleic acid. In various embodiments, the target nucleic acid molecule may be a portion of a gene, a regulatory sequence, genomic DNA, cDNA, RNA including mRNA and rRNA, or others.

Specifically, it is envisioned that the methods according to various embodiments described herein may be utilised in determining the presence or quantity of a target nucleic acid molecule for application in one or more of the following areas:
Detection of infectious diseases (e.g. COVID-19, Group B Streptococcus (GBS), sexually transmitted diseases, tuberculosis, identifying the causative agent of skin infections, distinguishing bacterial infections from viral infections etc);
Detection of cancer mutations;
SNP genotyping;
Food testing (e.g. do products genuinely contain components as claimed, such as poultry or seafood); and
Detection of pathogens in agriculture and aquaculture (e.g. pathogenic vs non-pathogenic Vibrio, white spot syndrome virus, iridovirus, koi herpes virus, scale drop disease virus, lates calcarifer herpes virus).

In various embodiments, the target nucleic acid molecule may be a nucleic acid of a pathogen, optionally a human pathogen, preferably a bacterial, fungal, parasite or viral nucleic acid molecule or a cDNA reverse transcript of a bacterial, fungal, parasite or viral RNA. In various embodiments, the target nucleic acid molecule may be a nucleic acid of a coronavirus, influenza virus, paramyxovirus or enterovirus. In various embodiments, the target nucleic acid molecule may be a nucleic acid of SARS-CoV-2 virus.

In various embodiments, the methods described herein may be readily adapted to detect any infectious agent or disease outbreak in the future, as well as being adaptable for other areas and uses that require detecting the presence, absence or quantity of a nucleic acid in a sample.

In particular, point-of-care or point-of-need detection methods according to various embodiments described herein may enable rapid, affordable, asset-light, simple-to-use and decentralized detection and assisted diagnosis of infectious diseases. For example, methods according to various embodiments described herein may be used for the detection and assisted diagnosis of infectious diseases, such as COVID-19, and permit testing to be exponentially scaled up around the world. This will help to limit human-to-human transmission of SARSCoV-2, so that communities can safely resume activities. RT-LAMP assays serve as one attractive class of point-of-need diagnostic tests. However, they are prone to false positives, especially if sequence-independent readouts like a pH-sensitive dye are employed. Unfortunately, most published or commercially available RT-LAMP assays for COVID-19 rely on such readouts, with colorimetric dyes being particularly popular.¹⁻⁵ Hence, there is a need to develop more reliable assays that incorporate an extra specificity checking step to guard against false positives. A few research groups have tried to address this issue, but their target detection strategies suffer from various shortcomings, including an absence of a separate probe from the LAMP primers, nonoptimal reaction conditions, and challenging probe or riboregulator design that likely requires several iterations of testing⁸⁻¹². Furthermore, the Proofman and oligonucleotide strand exchange (OSD) probe methods have not been validated with clinical samples.

Accordingly, the methods according to various embodiments described herein may enhance the specificity and sensitivity of RT-LAMP without compromising its speed.

In various embodiments, there is provided a detection probe that is designed specifically for use in the methods described herein. In various embodiments, the detection probe may be a single-stranded probe that recognises a probe binding site within the target nucleic acid and target amplicons. In particular, the detection probe may comprise a nucleic acid sequence complementary to the target nucleic acid and target amplicons, more particularly a region of the target nucleic acid that is amplified such that it is located in the amplicons formed by the isothermal amplification reaction.

Advantageously, the detection probe described herein is easy to design and may be used with any isothermal amplification setup, including LAMP. Importantly, there is no sequence context requirement of the detection probe and thus the detection probe described herein can be readily designed and its annealing temperature can be conveniently calculated using standard primer design software.

In various embodiments, the detection probe described herein does not have any probe binding site constraints and so may be placed at any available position on an amplicon. In various embodiments, the probe binding site may lie between the binding sites of primers used in the isothermal amplification reaction.

In various embodiments, said probe binding site may be different from and non-overlapping with any primer binding sites used in the isothermal amplification setup. In particular, the detection probe described herein may be designed to be separate and distinct from the primers to rule out spurious by-products and does not interfere with the process of isothermal amplification. That is, the detection probe described herein is a separate oligonucleotide and is not an extension of any primer used in the isothermal amplification reaction, and therefore is much less likely to interfere with the amplification process.

In various embodiments, the detection probe may be designed such that it can hybridize to the probe binding site on the amplicons formed under isothermal amplification assay conditions to form a double-stranded probe:target complex. The hybridization is typically achieved by designing the detection probe sequence such that the nucleotides contained therein can form Watson-Crick base pairs with the designated sequence of the probe binding site in the amplicons. Generally, when reference is made herein to “complementarity”, it is meant that the respective sequence can form Watson-Crick base pairs with its designated target or counterpart, however, “complementary” used herein is not restricted to mean “fully complementary” in that the respective sequence stretch does not have to be complementary over the entire length of the respective region, i.e. not all of the bases in the nucleotide sequence of the detection probe need to form Watson-Crick base pairs with its counterpart sequence of the probe binding site, so long as the probe can hybridize to the probe binding site.

Accordingly, the detection probe may comprise at least one deliberately mismatched base pairing with the sequence of the probe binding site in the amplicons, such that the detection probe may hybridize to the target nucleic acid or target amplicons with near-perfect complementarity except for the mismatched base pairing (i.e. not fully complementary).

In various embodiments, the detection probe may comprise at least one 3′ end (terminal) nucleotide mismatch, wherein the detection probe may hybridize to the target amplicons under isothermal amplification assay conditions except for the 3′ end (terminal) nucleotide mismatch and form a double-stranded probe:target complex.

In various embodiments, the at least one 3′ end nucleotide mismatch may comprise a single 3′ end nucleotide mismatch. In various embodiments, the single 3′ end nucleotide mismatch may be positioned 5 (MM5), 4(MM4), 3(MM3), 2(MM2), or 1(MM1) nucleotides away from the 3′ end of the detection probe. “MM” in this context is an abbreviation of “mismatch”. In various embodiments, the single 3′ end nucleotide mismatch may be positioned at the last (MM1) or second to last (MM2) nucleotide relative to the 3′ end of the detection probe. In various embodiments, the single 3′ end nucleotide mismatch may be positioned at the second to last (MM2) nucleotide relative to the 3′ end of the detection probe.

In various embodiments, the at least one 3′ end nucleotide mismatch may comprise two 3′ end nucleotide mismatches. In various embodiments, the two 3′ end nucleotide mismatches may be the last two nucleotides relative to the 3′ end of the detection probe (MM1+2).

In various embodiments, the detection probe may range in length from about 10 nucleotides to about 50 nucleotides, preferably about 12 to 30 nucleotides. In various embodiments, the detection probe is 17-30 nucleotide bases in length. In various embodiments, the detection probe is 17-25 nucleotide bases in length.

In various embodiments, the detection probe may be conjugated or attached to any fluorophore or quencher, more particularly any fluorophore-quencher pair. This is unlike the LUX primer and HyBeacon probe, where only a subset of fluorophores with self-quenching properties may be used.

The term “Fluorophore” as used herein, refers to a moiety that emits fluorescence when excited by light of the proper wavelength, whereas “quenchers” refer to a moiety that suppresses the fluorescence emission of the fluorophore. In a fluorescence resonance energy transfer (FRET) pair the two members of the pair influence each other as long as they are in close spatial proximity, for example when bound to the same molecule, with this influence becoming less pronounced the farther apart the two members are. This allows to detect the difference between an intact probe, where both moieties are in close proximity, and a cleaved probe, where each fragment comprises one member of the pair so that they are no longer in close proximity to each other. In a typically fluorophore-quencher pair, the quencher suppresses fluorescence of the fluorophore if both are present in the same molecule. Once both get separated by cleavage of the molecule such that both are no longer present in the same molecule, the influence of the quencher is reduced so that the fluorescence of the fluorophore is detectably increased.

In various embodiments, the detection probe may be conjugated or attached to a quencher-fluorophore pair at opposite ends of the probe and at a distance that allow the quencher to quench the fluorophore signal. The quencher-fluorophore pair may be positioned such that they may interact in the intact non-cleaved probe and selected such that the fluorescence signal changes upon cleavage of the probe. In the detection probes described herein, this is typically given, even if both are positioned on opposing 5′ and 3′ ends, respectively, of the probe, or vice versa.

In various embodiments, either the fluorophore or the quencher is attached to the 3′ end of the probe downstream of or at the site of the mismatch, that is, the fluorophore or the quencher may be conjugated to the last nucleotide at the 3′ end of the probe. In various embodiments, the quencher may be attached to the 5′ end of the detection probe and the fluorophore may be attached to the 3′ end of the detection. In various embodiments, the fluorophore may be attached to the 5′ end of the detection probe and the quencher may be attached to the 3′ end of the detection.

As the fluorophore or quencher may be conjugated to the 3′ end of the probe, it will be released from the probe after cleavage, thereby producing a fluorescent signal. In principle, the position of the fluorophore and quencher may also be swapped, in which case the quencher separates from the probe after cleavage. In particular, the 3′-end mismatched nucleotide may be cleaved off by a DNA polymerase after hybridization of the detection probe to the target amplicon, thereby separating the fluorophore and quencher from one another, which then results in a fluorescence signal.

In various embodiments, the DNA polymerase may be any DNA polymerase which possesses an inherent 3′-5′ exonuclease activity. In various embodiments, the DNA polymerase with 3′-5′ exonuclease activity may be a high-fidelity DNA polymerase. In various embodiments, the high-fidelity DNA polymerase may be selected from Q5 High-Fidelity DNA Polymerase (New England Biolabs), Platinum SuperFi II DNA Polymerase (Thermo Fisher), iProof High-Fidelity DNA Polymerase (Bio-Rad), HotStar HiFidelity DNA Polymerase (QIAGEN), Pfu DNA Polymerase (Vivantis Technologies), KOD-Plus-Neo (TOYOBO), Bst 2.0 DNA Polymerase, and Bsm DNA Polymerase (Thermo Fisher).

In various embodiments, the DNA polymerase may be in an amount in the range of 0.2 U to 1 U, preferably about 0.5 U (or 0.02 U/μL).

The cleavage of the detection probe at the 3′-end mismatch leads to the generation of a 3′-terminal (end) probe fragment that due to the lowered affinity for the target, in particular a lowered melting temperature of the 3′-terminal (end) probe fragment:target complex, cannot stay hybridized to the target under the isothermal amplification assay conditions. The released probe fragment may subsequently be detected and quantified by any suitable means known in the art. In various embodiments, the other probe fragment may remain hybridised to the target and be treated as a primer by the polymerase.

In various embodiments, the detection probe may be double quenched and comprise an internal quencher. The inclusion of an internal quencher and use of a double quenched probe may exhibit a significantly higher signal than a single quenched probe. In various embodiments, the internal quencher may be positioned at or close to the centre of the detection probe (i.e. attached close to or at the middle nucleotide relative to the length of the probe), such that the internal quencher does not interfere with hydridisation of the 3′-end but is still able to quench the fluorophore.

In various embodiments, there is provided the use of the detection probe as described herein for determining the presence or quantity of a target nucleic acid molecule in a sample using an isothermal amplification method. All embodiments disclosed above in relation to the detection probe and below in relation to the method described herein similarly apply to this use.

Accordingly, in various embodiments, there is provided a method for determining the presence or quantity of a target nucleic acid molecule in a sample using isothermal amplification, the method comprising:
(a) combining an isothermal amplification reaction mixture, a DNA polymerase with 3′-5′ exonuclease activity, and a detection probe with the sample (suspected of containing the target nucleic acid molecule), wherein the isothermal amplification reaction mixture comprises a primer set of at least two primers, wherein each primer recognises a distinct primer binding site within the target nucleic acid molecule, wherein the detection probe is a single-stranded probe that recognises a probe binding site within target amplicons, said probe binding site being different from and non-overlapping with any one of the primer binding sites, wherein the detection probe comprises at least one 3′ end nucleotide mismatch and a quencher-fluorophore pair at opposite ends of the probe at a distance that allow the quencher to quench the fluorophore signal, wherein either the fluorophore or the quencher are attached to the 3′ end of the probe downstream of or at the site of the mismatch, wherein the detection probe can hybridize to said target amplicons under isothermal amplification assay conditions except for the 3′ end nucleotide mismatch and form a double-stranded probe:target complex,
(b) amplifying the target nucleic acid molecule under isothermal amplification assay conditions that allow:
   i. generation of the target amplicons;
   ii. hybridization of the detection probe to the target amplicons to form the probe:target complex; and
   i. cleavage of the detection probe at the 3′ end nucleotide mismatch by the DNA polymerase with 3′-5′ exonuclease activity to release a 3′-terminal probe fragment comprising the quencher or fluorophore; and
(c) detecting and optionally quantifying the released probe fragments to determine the presence and optionally amount of the target nucleic acid molecule in the sample.

In various embodiments, the isothermal amplification may be selected from rolling circle amplification (RCA), loop-mediated isothermal amplification (LAMP), recombinase polymerase amplification (RPA), nucleic acid sequence based amplification (NASBA), transcription-mediated amplification (TMA), helicase dependent amplification (HDA), exponential amplification reaction (EXPAR), and strand displacement amplification (SDA).

In various embodiments, the detection probe may be added in an amount of 0.5-3 μM. In various embodiments, the detection probe may be added in an amount of 0.5-1 μM.

In various embodiments, the isothermal amplification may be loop-mediated isothermal amplification (LAMP). In this regard, the “target nucleic acid”, refers to the target nucleic acid to be detected but further encompasses the amplicons and concatemers produced by the LAMP reaction that include sequences of the target that are recognized by the inner primers, the loop primer(s) and the detection probe. Accordingly, when reference is made to a target that is bound by the LAMP primers or the detection probes, this term typically relates to the amplicons and concatemers as produced in the LAMP reaction, as these are more prevalent than the original target nucleic acid. “Amplicons” or “concatemers”, as used interchangeable herein, relate to the amplified products generated starting from the template, i.e. the original target nucleic acid, and dumbbell starting structure produced from the inner primers in a first part of the LAMP reaction. These structures contain multiple repeats of the relevant sequence elements described above.

As used herein, the terms “LAMP” or “loop-mediated isothermal amplification”, refer to a method that is performed at an essentially constant temperature without the need for a thermocycler. In LAMP, the target sequence is typically amplified at 60 to 65° C. using either two or three sets of primers (i.e. 4 to 6 primers) and a polymerase with high strand displacement activity in addition to a replication activity. DNA polymerase with strand displacement activity/properties is known to those skilled in the art as an ability of the polymerase to displace the downstream DNA strand encountered during synthesis along the target strand. Typically, 4 different primers are used to identify 6 distinct regions on the target gene, which adds highly to the specificity. An additional “loop primer” or pair of “loop primers” can further accelerate the reaction. Due to the specific nature of the action of these primers, the amount of DNA produced in LAMP is considerably higher than PCR based amplification. The LAMP method is described in U.S. Pat. Nos. 6,410,278 B1 and 7,374,913 B2. Generally, the method uses two inner primers (forward inner primer=FIP and backward inner primer=BIP), two outer primers (F3 and B3) which recognize six distinct regions in the target, and optionally one or two, preferably two, loop primers (loop forward=LF and/or loop backward=LB) to boost amplification efficiency. If two loop primers are used, one is preferably a loop forward primer and the other a loop backward primer. The inner primers comprise a target complementary region (typically referred to as F2 and B2) that facilitates hybridization and 5′ thereto a sequence that is identical to a sequence in the target nucleic acid located upstream (5′) relative to the sequence of the target bound by the target complementary region of the inner primer (typically referred to as F1c and B1c). Elongation of the inner primer by the polymerase thus creates a sequence comprising regions of self-complementarity in that the target-identical sequence on the 5′ end of the inner primer (B1c) can, after elongation, bind to the synthesized sequence downstream of the target-complementary region of the inner primer (referred to as B1) and act as a primer for further extension. The outer primers bind to a target region in the target nucleic acid that lies downstream (i.e. 3′) to the target region bound by the inner primers (referred to as F3c and B3c) and thus are responsible for the displacement of the elongated inner primer sequences from the template strand. The elongated inner primers are recognized and hybridized by the other primer of the inner primer pair and thus the dumbbell structured starting amplicons are generated. The dumbbell structures are then used for the following amplification, with the amplicons taking the form of concatemers. The principle of LAMP amplification is common general knowledge for those skilled in the art.

According, in various embodiments of the method described herein, and in accordance with the established principles of LAMP, the isothermal amplification reaction mixture may be a LAMP reaction mixture comprising a LAMP primer set of 4 to 6 primers, comprising two inner primers (FIP and BIP), two outer primers (F3 and B3), and optionally one or two loop primers (LF and/or LB). While it is known that the loop primer(s) increase(s) amplification efficiency, these are optional and not essential for carrying out the LAMP method. It is however preferred that one or two, preferably two, loop primers are included in the methods of the invention.

The two inner primers used in the methods thus may each comprise a target complementary region on their 3′ end (F2 and B2) and a target identical region on their 5′ end (F1c and B1c), where in the target nucleic acid the sequence (i.e. primer binding site) recognized by the target complementary region of the inner primers (termed F2c or B2c) lies 3′ to the sequence identical to the target identical sequence on the 5′ end of the inner primers (said sequence in the target termed F1c and B1c).

Accordingly, the two outer primers each comprise a target complementary region (F3 and B3), wherein in the target nucleic acid the sequence (i.e. primer binding site) targeted by the target complementary region of the outer primers (termed F3c and B3c) are located 3′ to the sequence of the target nucleic acid targeted by the target complementary region of the inner primers. This allows displacement of the elongated inner primers necessary for generation of the dumbbell shaped starting structures needed for concatemer formation in the later stages of LAMP.

The one or two optional loop primers each comprise a target complementary region that recognizes a sequence (i.e. primer binding site) between the target complementary region on the 3′ end of the inner primers or the complement thereof (i.e. the F2 or B2 region) and the sequence complementary to the target identical sequence on the 5′ end of the inner primers or the complement thereof (i.e. the F1 or B1 region). The forward loop primers preferably bind between F1 and F2. Similarly, preferred binding for the backward loop primers is thus between B1 and B2. It may be preferred that the loop primer set comprises loop primers that bind between the F1 and F2 and loop primers that bind between the B1 and B2 regions of the amplicons.

In various embodiments using LAMP as the isothermal amplification, two additional primer sets may be utilized in addition to the two inner primers (FIP and BIP) and two outer primers (F3 and B3), and two loop primers (LF and LB). These two additional primer sets may include stem primers, which target the single-stranded region in the center of the dumbbell structure, and the swarm primers, which hybridize to the template strand opposite to that of FIP or BIP so as to reveal the binding sites for the inner primers.

In various embodiments, the primer set may further comprise two swarm primers including a forward swarm primer and a backward swarm primer. In various embodiments, the primer set may further comprise two stem primers including a forward stem primer and a backward stem primer. In various embodiments, the primer set may further comprise two swarm primers and two stem primers.

In various embodiments, the respective binding sites recognized by the LAMP primers and the detection probe are non-overlapping. Accordingly, the probe binding site for the detection probe may be different from the primer binding site of the LAMP primers, more preferably non-overlapping with the LAMP primer binding sites.

The LAMP method is characterized by generating unique stem-loop structures, which contain single-stranded regions. These single-stranded regions may provide ideal positions for single strand probe hybridization without the need to separate the double-stranded DNA either through heating or strand displacement enzymes. LAMP is performed isothermally and probe hybridization has been optimized to be carried out at the same temperature. Both of these enable the LAMP reaction and probe hybridization to occur simultaneously, thus greatly facilitating real-time probe-mediated detection and improving the detection speed. Therefore, in the methods described herein hybridization probes may target sequences in the single-stranded loop regions.

Accordingly, in various embodiments, the probe binding site may be in a loop region of the target amplicons formed by the LAMP and may be different from and non-overlapping with any one of the primer binding sites. Binding of the detection probe in the loop region may ensure that probe:target hybridization does not interfere with the ongoing amplification reaction mediated by the inner primers.

Alternatively, in various embodiments, the probe binding site may be in a stem region of the target amplicons formed by the LAMP and may be different from and non-overlapping with any one of the primer binding sites.

In various embodiments, the target nucleic acid used as a template for the LAMP reaction may be any nucleic acid molecule. In various embodiments, the target nucleic acid molecule may be a nucleic acid of a pathogen, optionally a human pathogen, preferably a bacterial, fungal, parasite or viral nucleic acid molecule. In various embodiments, the target may be the viral RNA of SARS-CoV-2, more particularly an S-gene fragment of SARS-CoV-2 RNA.

In various embodiments, where the target is the S-gene of SARS-CoV-2 RNA, the primer set may include:
(1) the LAMP inner forward primer may comprise the nucleic acid sequence set forth in SEQ ID NO:3 or 4 (S2 FIP and S2 FIP (−1nt)) or a variant thereof having at least 90% sequence identity over the entire length;
(2) the LAMP inner backward primer comprises the nucleic acid sequences set forth in SEQ ID NO: 5 or 6 (S2 BIP and S2 BIP (−1nt)) or a variant thereof having at least 90% sequence identity over the entire length;
(3) the LAMP outer forward primer comprises the nucleic acid sequence set forth in SEQ ID NO:1 (S2 F3) or a variant thereof having at least 90% sequence identity over the entire length;
(4) the LAMP outer backward primer comprises the nucleic acid sequence set forth in SEQ ID NO:2 (S2 B3) or a variant thereof having at least 90% sequence identity over the entire length;
(5) the LAMP loop forward primer, if present, comprises the nucleic acid sequence set forth in SEQ ID NO:7 (S2 LF) or a variant thereof having at least 90% sequence identity over the entire length; and
(6) the LAMP loop backward primer, if present, comprises the nucleic acid sequence set forth in SEQ ID NO:8 (S2 LB) or a variant thereof having at least 90% sequence identity over the entire length.

In various embodiments, where the target is the S-gene of SARS-CoV-2 RNA, the primer set may further include:
(7) the LAMP Swarm forward primer, if present, comprises the nucleic acid sequence set forth in SEQ ID NO:9 (S2 Swarm F1c) or a variant thereof having at least 90% sequence identity over the entire length; and
(8) the LAMP Swarm backward primer, if present, comprises the nucleic acid sequence set forth in SEQ ID NO:10 (S2 Swarm B1c) or a variant thereof having at least 90% sequence identity over the entire length.

In various embodiments of the methods for SARS-CoV-2 detection, the detection probe may comprise the nucleic acid sequence set forth in any one of SEQ ID Nos. 25-27 and 35-46 or a variant thereof having at least 90% sequence identity over the entire length. SEQ ID NO: 25, 26 and 35-40 may be for targeting a stem region, while SEQ ID NO:27 and 41-46 may be for targeting a loop region. In various embodiments, the detection probe may comprise the nucleic acid sequence set forth in any one of SEQ ID Nos. 25-27 and 35-46 with a quencher conjugated to the 5′ end and a fluorophore conjugated to the 3′end, whereby the quencher may be IABkFQ and the fluorophore may be FAM. In various embodiments, the detection probe may further comprise an internal quencher, whereby the internal quencher may be a ZEN quencher.

When reference is made to sequence identity, this means that in a given nucleic acid molecule the respective nucleotide at a given position is identical to the nucleotide in a reference nucleic acid molecule at the corresponding position. The level of sequence identity is given in % and can be determined by an alignment of the query sequence with the template sequence.

The determination of the identity of nucleotide sequences is achieved by a sequence comparison. This comparison or alignment is based on the BLAST algorithm well-established and known in the art and is in principle carried out by aligning stretches of nucleotides in the nucleotide sequences with each other. Another algorithm available in the art is the FASTA algorithm. Sequence comparisons (alignments), in particular multiple sequence comparisons, can be generated using computer programs. Commonly used are for example the Clustal series or programs based thereon or the respective algorithms. Further possible are sequence comparisons (alignments) with the computer program Vector NTI® Suite 10.3 with the pre-set standard parameters, the AlignX-module of which is based on ClustalW. If not explicitly defined otherwise, sequence identity is determined using the BLAST algorithm.

Such a comparison allows determining the identity of two sequences and is typically expressed in % identity, i.e. the portion of identical nucleotides in the same or corresponding positions. If not explicitly stated otherwise, the sequence identities defined herein relate to the percentage over the entire length of the respective sequence, i.e. typically the reference sequence. If the reference sequence is 20 nucleotides in length, a sequence identity of 90% means that 18 nucleotides in a query sequence are identical while 2 may differ.

In various embodiments, step (a) may further comprise pyrophosphatase. In various embodiments, the pyrophosphatase may be in an amount in the range of 0.2 U to 1 U, preferably about 0.5 U (or 0.02 U/μL). In various embodiments, the pyrophosphatase may be a thermostable inorganic pyrophosphatase (TIPP). In various embodiments, the pyrophosphatase may be Inorganic pyrophosphatase (PPase).

In various embodiments, the background noise in the method described herein is low in the absence of the intended target nucleic acid being in the sample. In particular, if the detection probe described herein does not find its intended target, the fluorophore and quencher will remain intact on the same molecule and there will be minimal fluorescence signal regardless of oligonucleotide structure. In contrast, probes such as molecular beacons used in existing methods are designed to be non-fluorescent only when they are in a double-stranded conformation. As there is a delicate balance between hairpin stability and target hybridization, existing methods relying on such probes often encounter delayed fluorescence signals when the hairpin is too stable or high background noise when the hairpin is prone to melting, especially in LAMP where the operating temperature of 65° C. is relatively high. This was evaluated using actual clinical samples with a range of viral loads and it was found that it exhibited performance characteristics (specificity of 95% and a LoD of 8 copies per reaction) that were similar to those of many reported RT-qPCR assays for COVID-19, giving confidence that the method described herein could work in real-life scenarios.

Besides fluorescence readouts, in various embodiments, a lateral flow readout may also be used to detect cleavage of detection probes. In such embodiments, the detection probe may be labelled on both ends with markers that are recognized by antibodies. Examples of such markers include, without limitation, antigens including fluorescent markers that simultaneously function as antigen. Concrete examples include, without limitation, biotin, FITC and digoxigenin. In the lateral flow detection, the sample may be typically run on a capillary bed after being put on a first element of the lateral flow strip, the so-called sample pad. It then migrates to the second element, typically the conjugate pad is typically stored the so-called detection conjugates, for example in a dried format together with a matrix that allows the binding reaction between the target molecule (e.g., an antigen) and its chemical partner (e.g., antibody) that has been immobilized. As the sample fluid dissolves the conjugates and the matrix, the sample and conjugate mix while flowing through the porous structure. In this way, the analyte binds to the detection conjugates while migrating further through the capillary bed. This material has one or more areas (often called stripes) where a third or further “capture” molecule has been immobilized. By the time the sample-conjugate mix reaches these stripes, analyte has been bound by the detection conjugates and the “capture” molecule binds the complex. After a while, when more and more fluid has passed the stripes, complexes accumulate and the stripe-area changes color. Typically, there are at least two stripes: one (the control) that captures any detection conjugate and thereby shows that reaction conditions and technology worked fine and one that contains a specific capture molecule and only captures those conjugates which are complexed with an analyte molecule. After passing these reaction zones, the fluid enters the final porous material, the wick, that simply acts as a waste container.

In various embodiments, each lateral flow strip may comprise gold-conjugated IgG antibodies against the fluorophore near the sample pad, antibodies against the quencher immobilized at the control line, and antibodies against IgG immobilized at the test line. In the case of a target-free sample, the probe will stay intact such that when the reaction is loaded on the strip, the gold-conjugated IgG first binds to the fluorophore and then the entire IgG-probe complex is captured at the control line. Consequently, a dark band is observed only at the control line. However, in the case of a sample containing the target, the polymerase will cleave off the fluorophore such that when the reaction is loaded on the strip, the gold-conjugated IgG still binds to the fluorophore, but now, some of the gold will not be deposited at the control line as the fluorophore is free. Instead, the IgG-fluorophore complex continues flowing along the strip to the test line, where it is captured by the anti-IgG antibodies. Consequently, a dark band is observed at the test line.

The method described herein is thus compatible with both a fluorescent readout and a lateral flow readout. In contrast, existing detection methods are only capable of providing a fluorescent readout. Accordingly, in various embodiments, the detection method in step (c) may be lateral flow detection and/or fluorescence detection.

In various embodiments, the method described herein is also straightforward to utilize for multiplex detection of several different targets. In particular, the method described herein can be readily deployed for simultaneous detection of multiple distinct targets simply by the use of two or more detection probes described herein with different fluorophore-quencher combinations. This is unlike existing methods such as PEI-LAMP, where it is difficult to interpret a mixture of colours within the precipitate. For example, the method described herein can detect both SARS-CoV-2 and a human internal control in the same reaction tube by using two different fluorophores.

Accordingly, the method described herein may be adapted into a multiplexing method and used for determining the presence or quantity of two or more target nucleic acid molecules in the sample, wherein the method uses two or more primer sets and two or more detection probes designed for multiple target nucleic acid molecules.

In various embodiments, the method described herein may be a multiplexing method and is for determining the presence, absence and optionally amount of two or more target nucleic acid molecules in the sample, wherein the method uses one or more primer sets and/or one or more detection probes as described herein for each target nucleic acid molecule or for multiple related target nucleic acid molecules.

In various embodiments, the method described herein can be readily used to detect point mutations and single nucleotide variations (SNVs). Existing methods lack the intrinsic property to resolve single nucleotide differences within the target sequence. As the detection probe described herein requires the 3′ end to be mismatched, this provides an opportunity to identify single nucleotide variations in the amplicon. This feature is useful not only for the identification of wild type viruses but also for the detection of new viral variants that emerge over the course of a pandemic. In particular, the method according to various embodiments described herein may be used to formulate a rapid, sensitive, and highly specific diagnostic assay for the identification of COVID-19.

In another aspect, there is provided a kit for determining the presence or quantity of a target nucleic acid molecule in a sample using isothermal amplification, the kit comprising:
a) an isothermal amplification reaction mixture;
b) a DNA polymerase with 3′-5′ exonuclease activity; and
c) a detection probe.

The components a)-c) are, in various embodiments, defined as described above for the identical components in relation to the methods described herein.

In various embodiments, the kit may further comprise pyrophosphatase. In various embodiments, the pyrophosphatase may be a thermostable inorganic pyrophosphatase (TIPP). In various embodiments, the pyrophosphatase may be Inorganic pyrophosphatase (PPase).

All embodiments disclosed above in relation to the methods described herein and detection probe described herein similarly apply to the kit.

### EXAMPLES

### Materials and Methods

Synthesis of Synthetic Viral RNA. For SARS-CoV-2, the S-gene fragment was amplified by PCR from a plasmid that was previously generated¹³ using Q5 High-Fidelity DNA Polymerase (New England Biolabs). To enable in vitro transcription (IVT), the forward primer was appended at the front with the T7 promoter sequence (5′-TAATACGACTCACTATAGG-3′). Amplified products were gel extracted with the PureNA Biospin Gel Extraction Kit (Research Instruments). At least 50 ng of T7-containing PCR product was used as template for IVT with the HiScribe T7 Quick High Yield RNA Synthesis Kit (New England Biolabs). The reaction was incubated overnight at 37° C. for maximum yield. Following Dnase I digestion for 1 h, the RNA was purified with the RNA Clean & Concentrator-5 Kit (ZYMO Research), analyzed by 2% TAEagarose gel electrophoresis to assess RNA integrity, quantified with NanoDrop 2000, and stored at −20° C. The concentration values obtained from NanoDrop correlated very well with those obtained from Qubit. For other viruses tested in the specificity experiments, RNAs from the Respiratory Virus Research Panel (Twist Biosciences) were used.

RT-LAMP Reaction with LANTERN Probes. All reactions were set up in a dedicated clean biosafety cabinet that was UV irradiated before every use. Synthetic SARS-CoV-2 RNA templates were serially diluted and amplified using the WarmStart LAMP Kit (New England Biolabs). Similar to previous work,¹³ 10×S-gene LAMP primer mix was prepared with concentration of 2 μM for F3, 4 μM for B3, 8 μM for FIP(PM), BIP(PM), FIP(tPM-3), BIP(tPM-3), LF, and LB, and 16 μM for swarm F1c and swarm B1c. The RTLAMP reaction was set up with 12.5 μL WarmStart LAMP Mastermix, 2.5 μL 10×S-gene primer mix, 2.5 μL 0.4 μM guanidine HCl, 0.25 μL thermostable inorganic pyrophosphatase (New England Biolabs), 0.25 μL Q5 high-fidelity DNA polymerase (New England Biolabs), 0.125 μL 100 μM LANTERN probe against S-gene, 5 μL synthetic RNA, and RNase-free water such that the total reaction volume was 25 μL. For reactions with an internal control incorporated, 0.75 μL 10×LAMP primers (2 μM for F3 and B3, 16 μM for FIP and BIP, and 8 μM for LF and LB) and 0.125 μL 100 μM LANTERN probe for human ACTB were also added to the reaction mix. Subsequently, each sample tube was incubated for 40 min at 65° C. using a CFX96 Real-Time PCR Detection System (Bio-Rad) with fluorescence in the FAM or Cy5 channel measured every minute. The RFU reported was the raw default output from the instrument's CFX Maestro Software. The Ct value was computed automatically by the software from the time when the fluorescent signal increased significantly above background, which is when the amplification is at the beginning of the exponential phase. Besides Q5, the other high-fidelity DNA polymerases that were tested included Platinum SuperFi II DNA Polymerase (Thermo Fisher), iProof High-Fidelity DNA Polymerase (Bio-Rad), HotStar HiFidelity DNA Polymerase (QIAGEN), Pfu DNA Polymerase (Vivantis Technologies), and KOD-Plus-Neo (TOYOBO). In addition, besides the WarmStart LAMP Kit, which utilized a Bst 2.0 DNA Polymerase, the Bsm DNA Polymerase (Thermo Fisher) was also tested with the LANTERN probes, according to manufacturer's instructions. All LAMP primers and LANTERN probes used are given in Tables 1, 2 and 3.

TABLE 1
List of Primers

| | SEQ ID | |
|---|---|---|
| Name | NO | Primer Sequence |
| S2 F3 | 1 | TTAATTTAGTGCGTGATCTCC |
| S2 B3 | 2 | AGCATCTGTAATGGTTCCAT |
| S2 FIP | 3 | TGTAAAGCAAGTAAAGTTTGAA |
| | | ACCCTCAGGGTTTTTCGGCT |
| S2 FIP (−1 nt) | 4 | TGTAAAGCAAGTAAAGTTTGAA |
| | | ACCCTCAGGGTTTTTCGGC |
| S2 BIP | 5 | TGGACAGCTGGTGCTAATAGAA |
| | | AAGTCCTAGGTTGAAG |
| S2 BIP (−1 nt) | 6 | TGGACAGCTGGTGCTAATAGAA |
| | | AAGTCCTAGGTTGAA |
| S2 LF | 7 | GGCAAATCTACCAATGGTTCTA |
| | | A |
| S2 LB | 8 | GCAGCTTATTATGTGGGTTAT |
| S2 Swarm F1c | 9 | TGTAAAGCAAGTAAAGTTTGAA |
| | | ACC |
| S2 Swarm B1c | 10 | TGGACAGCTGGTGCT |
| ACTB Set2 F3 | 11 | GGCATCCACGAAACTACCTT |
| ACTB Set2 B3 | 12 | GCCGATCCACACGGAGTAC |
| ACTB Set2 FIP | 13 | TGCCGCCAGACAGCACTGTGTG |
| | | AAGTGTGACGTGGACATC |
| ACTB Set2 BIP | 14 | TTGCCGACAGGATGCAGAAGGG |
| | | CGCTCAGGAGGAGCAAT |
| ACTB Set2 LF | 15 | GGCGTACAGGTCTTTGCG |
| ACTB Set2 LB | 16 | CCTGGCACCCAGCACAAT |
| ACTB Set2 | 17 | TGCCGCCAGACAGCACTGTG |
| Swarm F1c | | |
| ACTB Set2 | 18 | TTGCCGACAGGATGCAGAAGG |
| Swarm B1c | | |
| GAPDH Set3 F3 | 19 | TCATCTCTGCCCCCTCTG |
| GAPDH Set3 B3 | 20 | TCTTCTGGGTGGCAGTGA |
| GAPDH Set3 FIP | 21 | GTGCAGGAGGCATTGCTGATGA |
| | | CTGATGCCCCCATGTTCG |
| GAPDH Set3 BIP | 22 | CCAACTGCTTAGCACCCCTGGT |
| | | GGCATGGACTGTGGTCA |
| GAPDH Set3 LF | 23 | GTTGTCATACTTCTCATGGTTC |
| | | AC |
| GAPDH Set3 LB | 24 | CATCCATGACAACTTTGGTATC |
| | | G |

TABLE 2
List of individual components of probes
(underlined nucleotides reflect mismatches)

| | 5′ | Internal | 3′ | SEQ | |
|---|---|---|---|---|---|
| Name | moiety | Quencher | moiety | ID NO | Probe Sequence |
| S2 Stem MM1 | IABKFQ | — | FAM | 25 | CTCCTGGTGATTCTTCTTCAGGa |
| SQ | | | | | |
| S2 Stem MM1 | IABKFQ | ZEN | FAM | 25 | CTCCTGGTGATTCTTCTTCAGGa |
| DQ | | | | | |
| S2 Stem | IABKFQ | ZEN | FAM | 26 | CTCCTGGTGATTCTTCTTCAGca |
| MM1+2 DQ | | | | | |
| S2 LoopB | IABKFQ | ZEN | FAM | 27 | CAGCTTATTATGTGGGTTATgg |
| MM1+2 DQ | | | | | |
| ACTB LoopB | Cy5 | — | IAbRQSp | 28 | GGTGCCAGGGCAGTGATa |
| MM1 SQ | | | | | |
| ACTB Stem | Cy5 | — | IAbRQSp | 29 | TGCCAGGGTACATGGTGc |
| MM1 SQ | | | | | |
| ACTB LoopB | Cy5 | — | IAbRQSp | 30 | GGTGCCAGGGCAGTGATag |
| MM1+2 SQ | | | | | |
| ACTB LoopB | Cy5 | — | IAbRQSp | 31 | TGCCCTGGCACCCAGCACAca |
| site2 MM1+2 | | | | | |
| SQ | | | | | |
| ACTB LoopF | Cy5 | — | IAbRQSp | 32 | TGGCGTACAGGTCTTTGCtt |
| MM1+2 SQ | | | | | |
| ACTB Stem | Cy5 | — | IAbRQSp | 33 | TGCCAGGGTACATGGTGca |
| MM1+2 SQ | | | | | |
| ACTB Stem | Cy5 | — | IAbRQSp | 34 | CCACCATGTACCCTGGgt |
| site2 MM1+2 | | | | | |
| SQ | | | | | |

ZEN = internal ZEN™ quencher, FAM = 6-carboxyfluorescein, IABKFQ = lowa Black® FQ quencher; IAbRQSp = lowa Black® RQ quencher, Cy5 = Indodicarbocyanine-5.

TABLE 3
List of Complete Probes of Table 2
(underlined nucleotides reflect mismatches)

| Name | Probe |
|---|---|
| S2 Stem MM1 SQ | IABkFQ-CTCCTGGTGAT |
| | TCTTCTTCAGGa-FAM |
| S2 Stem MM1 DQ | IABkFQ-CTCCTGGT/ZEN/ |
| | GATTCTTCTTCAGGa-FAM |
| S2 Stem MM1+2 DQ | IABkFQ-CTCCTGGT/ZEN/ |
| | GATTCTTCTTCAGca-FAM |
| S2 LoopB MM1+2 DQ | IABkFQ-CAGCTTATT/ |
| | ZEN/ATGTGGGTTATgg- |
| | FAM |
| ACTB LoopB MM1 SQ | Cy5-GGTGCCAGGGCAGTG |
| | ATa-IAbRQSp |
| ACTB Stem MM1 SQ | Cy5-TGCCAGGGTACATGG |
| | TGc-IAbRQSp |
| ACTB LoopB MM1+2 SQ | Cy5-GGTGCCAGGGCAGTG |
| | ATag-IAbRQSp |
| ACTB LoopB site2 MM1+2 SQ | Cy5-TGCCCTGGCACCCAG |
| | CACAca-IAbRQSp |
| ACTB LoopF MM1+2 SQ | Cy5-TGGCGTACAGGTCTT |
| | TGCtt-IAbRQSp |
| ACTB Stem MM1+2 SQ | Cy5-TGCCAGGGTACATGG |
| | TGca-IAbRQSp |
| ACTB Stem site2 MM1+2 SQ | Cy5-CCACCATGTACCCTG |
| | Ggt-IAbRQSp |

Evaluation of LANTERN Assay with Contrived Swab and Saliva Samples. Heat-inactivated SARS-CoV-2 (ATCC VR-1986HK) was serially diluted into clinically negative UTM (Copan) or healthy donor saliva. 8.3 μL of sample at each dilution was treated with 1 μL Proteinase K (New England Biolabs) and vortexed for 1 min at room temperature. The treated sample was then heated at 95° C. for 5 min before 2 μL was used for RT-LAMP. Alternatively, 500 μL healthy donor saliva was added to 500 μL ZeroPrep Lysis Buffer (Veredus) before SARS-CoV-2 virions at different dilutions were spiked in. The contrived specimens using this commercially available saliva collection kit were processed according to manufacturer's instructions.

Evaluation of LANTERN Assay with Clinical RNA Samples. Nasopharyngeal and/or throat swab samples were collected from COVID-19 suspected patients in Viral Transport Media (VTM) (MP Biomedicals, USA) obtained from the Institute for Urban Disease Control and Prevention (IUDC), Thailand. Viral RNA was extracted from 200 μL of each swab specimen using a magLEAD 12gC instrument with a magLEAD Consumable Kit (Precision System Science, Japan) following manufacturer's instructions. SARS-CoV-2 detection was confirmed by Allplex 2019-nCOV Assay (Seegene, Korea), 14 whereby a Ct value of 35.5 (based on N-gene) was estimated to be equivalent to 4 copies of the virus. Each LANTERN-LAMP reaction was set up with 1 μL of extracted RNA. Ethics approval for the use of clinical samples was given by the Institutional Review Board of the Faculty of Medicine at Chulalongkorn University (IRB Number 302/63).

### Results and Discussion

### Example 1: Development of a Sequence-Specific Probe for RTLAMP

Initially, examination of how the fluorescence signal is generated in RT-qPCR assays was investigated. There are three main approaches, namely SYBR Green, LightCycler probes, and TaqMan probe. The commonly used SYBR Green dye binds to generic DNA and cannot be used to identify a specific sequence. LightCycler probes have previously been deployed in a LAMP-based diagnostic test,⁵⁹ but they are challenging to position on short amplicons or use in multiplex assays. Lastly, the TaqMan methodology leverages on the 5′-3′ exonuclease activity of Taq polymerase to digest a probe that is hybridized to the target amplicon. As the probe is labelled with a fluorophore and a quencher at the 5′ and 3′ end respectively, Taq-mediated cleavage causes the fluorophore to be released from the probe, thereby generating a signal. However, TaqMan probes are not directly applicable to LAMP due to the absence of 5′-3′ exonuclease activity in the Bst DNA polymerase used in the isothermal amplification reaction.

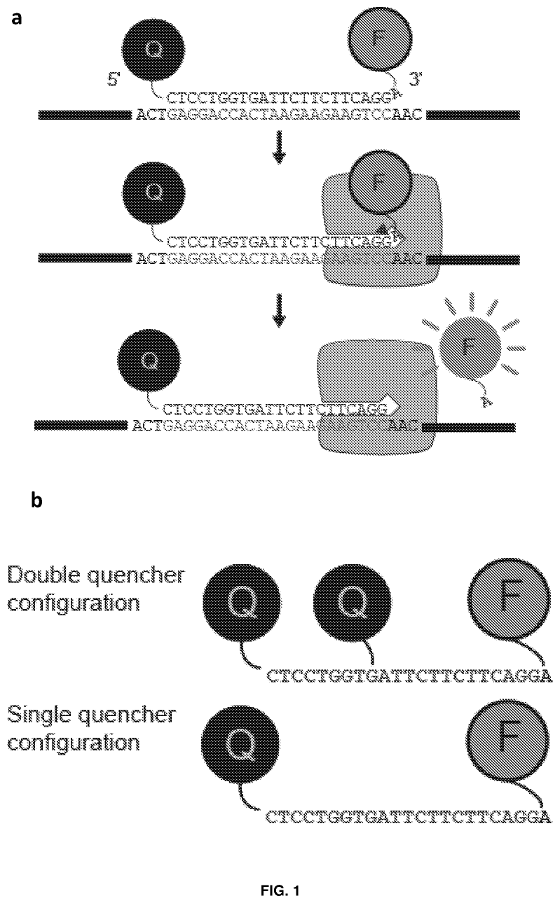

To adapt the TaqMan methodology for sequence-specific detection in LAMP assays, the probe was modified to carry a single nucleotide mismatch at the 3′ end of the probe and introduce it along with a high-fidelity DNA polymerase, which possesses an inherent 3′-5′ exonuclease activity for proofreading (FIG. 1a). It was rationalized that in the presence of the intended amplicon, the probe will hybridize to the target with near perfect complementarity except for the 3′ end mismatch, which will then be cleaved off by the proofreading DNA polymerase. Consequently, as the fluorophore is conjugated to the last nucleotide of the probe, it will be released from the probe after cleavage, producing a fluorescent signal. In principle, the position of the fluorophore and quencher may also be swapped, in which case the quencher separates from the probe after cleavage of the mismatched nucleotide. Hence, this method will enable sequence-specific detection in an isothermal amplification reaction via exonuclease digestion in a similar manner to the TaqMan approach. This was named as Luminescence from Anticipated Target due to Exonuclease Removal of Nucleotide mismatch (LANTERN).

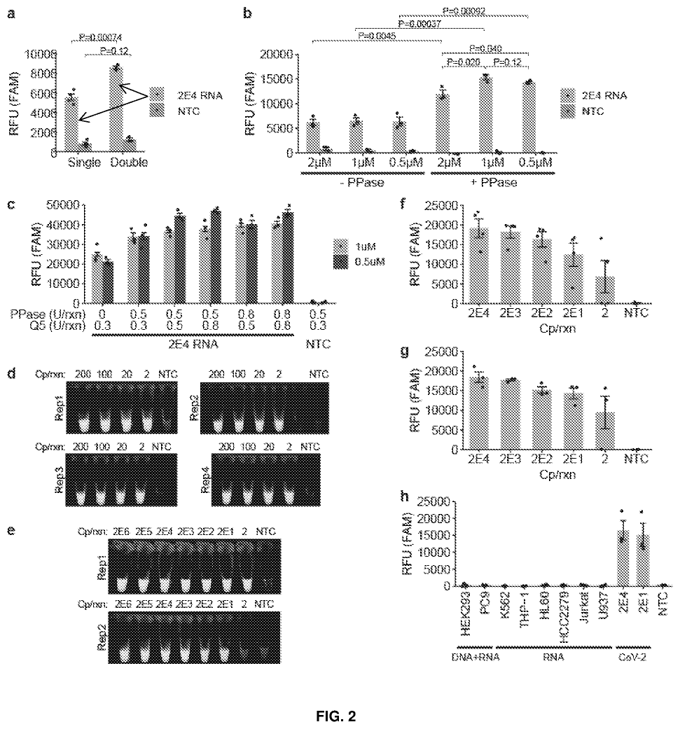

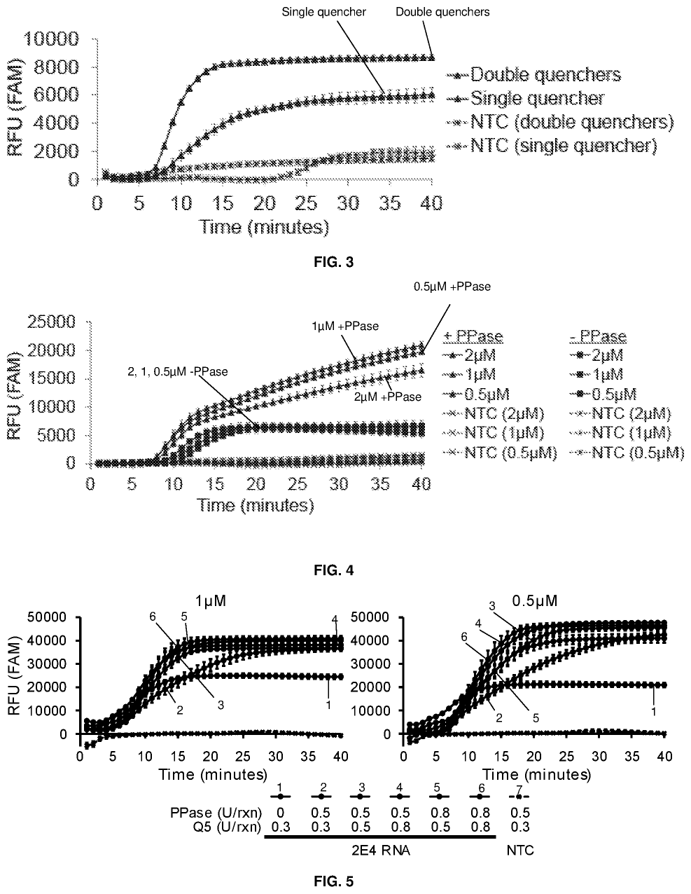

As an initial proof of concept, RT-LAMP was performed on synthetic SARS-CoV-2 RNA using primers against the Sgene¹³ and tested probes targeting the stem region of the amplicon. The probes were labelled with FAM at their 3′ end and were conjugated with either one or two quenchers toward the 5′ end (FIG. 1b). Consistent with the hypothesis that there was a strong increase in fluorescence over time in the presence of the viral template, with the double quenched probe exhibiting a significantly higher signal than the single quenched probe (P<0.001, two-sided Student's t test) (FIG. 2 and FIG. 3).

A large amount of pyrophosphate is generated in LAMP reactions, causing magnesium to precipitate out of solution. Consequently, the concentration of magnesium ions available for enzymatic activity is reduced over time. The pyrophosphate itself might also be inhibitory. Hence, the addition of a thermostable pyrophosphatase was tested to see if it would improve the assay with the double quenched probe. Overall, it was found that the addition of 0.5 U pyrophosphatase increased the fluorescence intensity significantly (P<0.01, Student's t test), regardless of the probe concentration (FIG. 2b and FIG. 4). Additionally, 0.5-1 μM of the double quenched probe appeared to be optimal with pyrophosphatase as they produced the highest fluorescence signal with minimal background. Moreover, it was observed that increasing the amount of Q5 high-fidelity DNA polymerase from 0.3 U to 0.5-0.8 U further enhanced the fluorescence signal and reaction kinetics (FIG. 2c and FIG. 5).

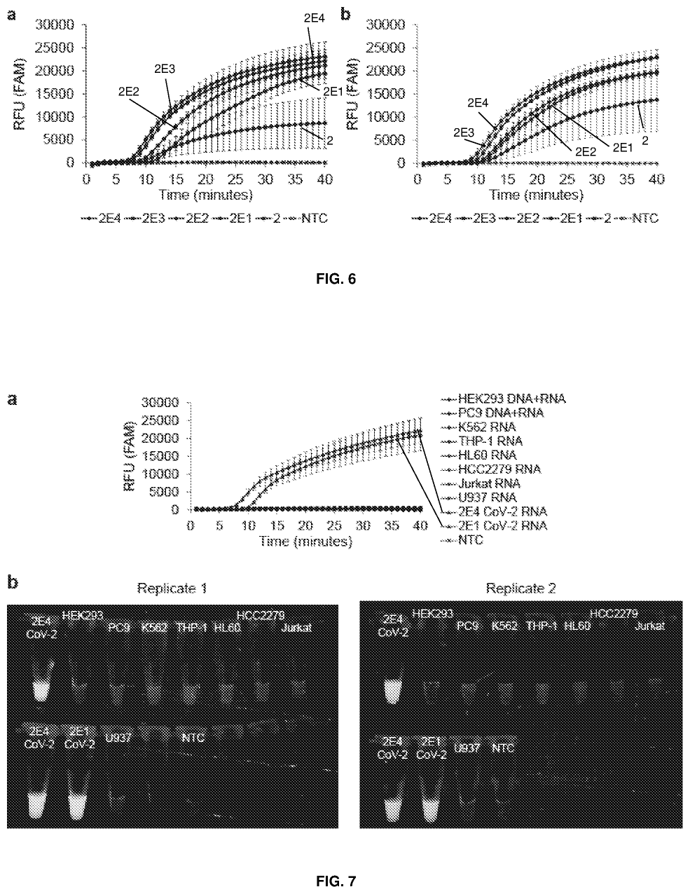

Next, the sensitivity of the assay. To this end, RT-LAMP was performed on variable copies of synthetic SARS-CoV-2 RNA with a double quenched probe and the addition of a pyrophosphatase and Q5 high-fidelity DNA polymerase. First, it was visualized that the sample tubes on a portable gel illuminator after 25 min of reaction and observed that 20 copies of template could be reliably detected with either 0.5 U (FIG. 2d) or 0.8 U (FIG. 2e) of each extra enzyme. Second, the fluorescence signal was monitored in a real-time PCR instrument and confirmed that the analytical limit of detection (LoD) of the assay was 20 copies per reaction in the absence (FIG. 2f and FIG. 6a) or presence (FIG. 2g and FIG. 6b) of heat-inactivated human saliva. It was further verified that the LAMP primers for SARS-CoV-2 did not cross-hybridize with any human RNA or DNA (FIG. 2h and FIG. 7).

### Example 2: Incorporation of a Human Internal Control within the Same Reaction

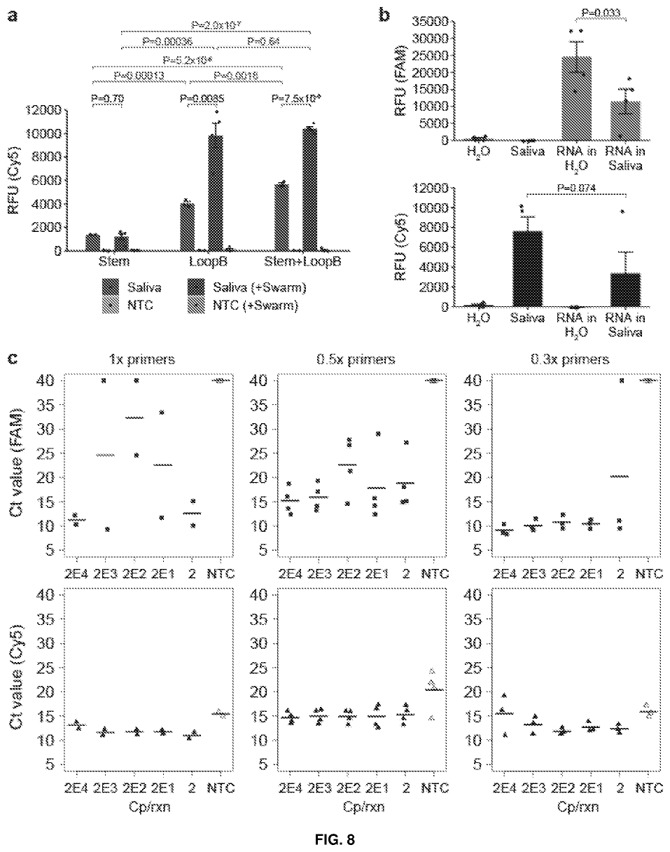

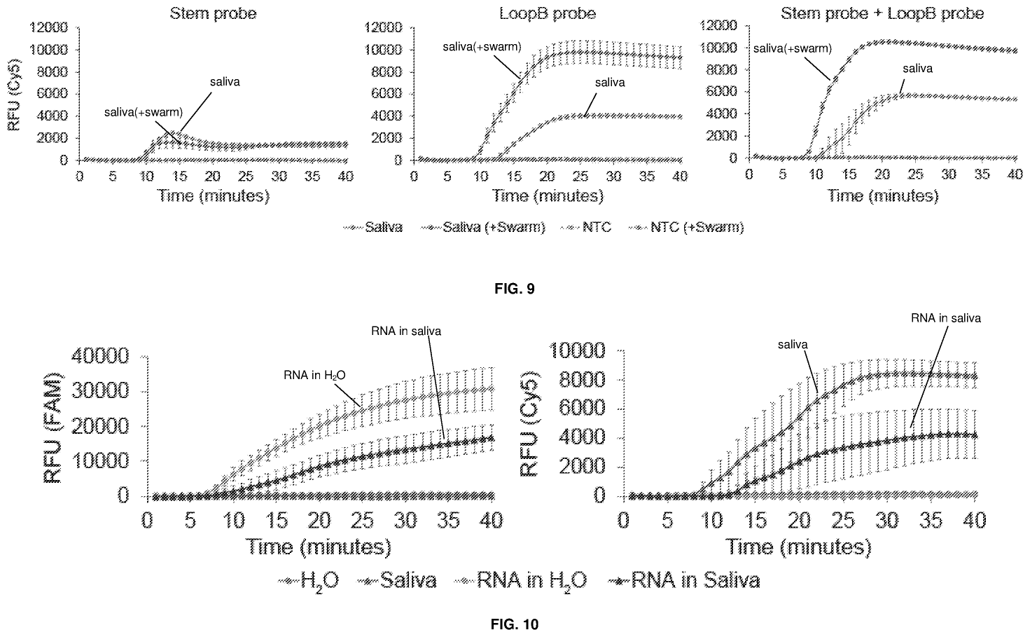

A human internal control is required for a diagnostic assay to ascertain that any negative result is due to an absence of the virus and not merely due to insufficient sample input. A set of LAMP primers targeting the human ACTB gene that is compatible with a set of LAMP primers targeting the viral Sgene was previously designed and validated.¹³ Hence, two Cy5-conjugated LANTERN probes were designed against the ACTB amplicon and evaluated them on heat-inactivated human saliva with or without additional swarm primers for the LAMP reaction (FIG. 8a and FIG. 9). Although both probes gave clear signals only in the presence of saliva, the fluorescence intensity from the loopB-targeting probe was significantly higher than that from the stem-targeting probe (P<0.001, one-sided Student's t test). Addition of swarm primers to the reaction with loopB probe further enhanced the fluorescence signal. Consequently, in all subsequent ACTB LAMP reactions, the swarm primers were included and the loopB probe utilized to detect the human amplicon specifically.

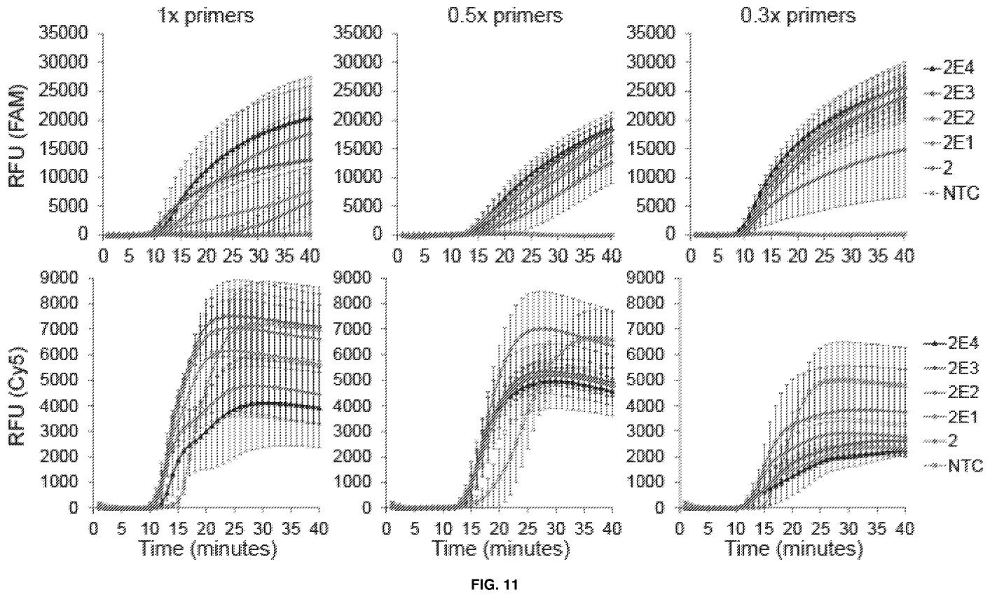

Next, it was evaluated whether the LANTERN assay could be multiplexed by combining the human ACTB and viral S-gene LAMP primers and probes together into a one-pot reaction. Application of the one-pot setup on 20 000 copies of synthetic SARS-CoV-2 RNA spiked into heat-inactivated saliva revealed that both the viral S-gene and human ACTB could be simultaneously detected (FIG. 8b and FIG. 10). However, the fluorescence signal was appreciably lower in the multiplex reaction than in a singleplex reaction (viral RNA alone or saliva alone), suggesting that there might be some competition between the S-gene and ACTB primers and probes. Then decreasing copies of SARS-CoV-2 RNA were tested in saliva (FIG. 8c and FIG. 11). When the S-gene primers and ACTB primers were present in equal amounts, it was observed that the assay encountered difficulty in detecting 2000 or less copies of viral RNA reliably. Lowering the amount of ACTB primers by 3-fold restored assay sensitivity for SARS-CoV-2 to 20 copies per reaction, although there was a concomitant drop in overall fluorescence intensity in the Cy5 channel. Nevertheless, 0.3×ACTB primers was proceeded with, since sensitivity for the virus is a key performance metric for a COVID-19 test.

### Example 3: Optimization and Further Characterization of LANTERN Probes

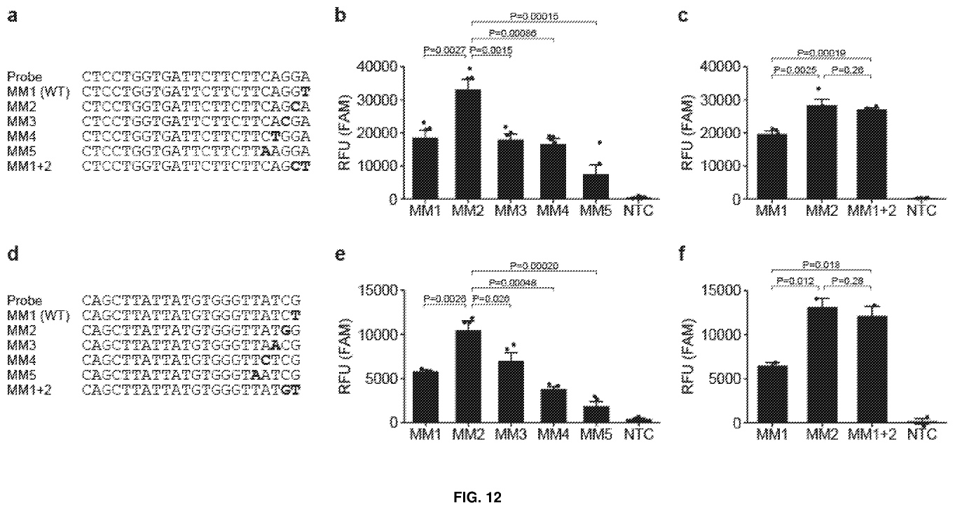

So far, the single nucleotide mismatch has been placed between probe and template at the 3′ end, but it was investigated how shifting the mismatch position inward would alter the fluorescence signal. To this end, a series of synthetic SARS-CoV-2 (S2) templates were generated whereby a mismatch occurred at variable positions along the binding site of the double quenched probe, which targeted the stem region of the amplicon (FIG. 12a). The stem targeting probes and their sequences are listed in Table 4.

TABLE 4
List of probe sequences used (bold and
underlined font indicating the
mismatch nucleotide)

| | SEQ ID | |
|---|---|---|
| Name | NO | Sequence |
| Probe | 25 | CTCCTGGTGATTCTTCTTCAGGA |
| MM1 (WT) | 35 | CTCCTGGTGATTCTTCTTCAGGT |
| MM2 | 36 | CTCCTGGTGATTCTTCTTCAGCA |
| MM3 | 37 | CTCCTGGTGATTCTTCTTCACGA |
| MM4 | 38 | CTCCTGGTGATTCTTCTTCTGGA |
| MM5 | 39 | CTCCTGGTGATTCTTCTTAAGGA |
| MM1+2 | 40 | CTCCTGGTGATTCTTCTTCAGCT |

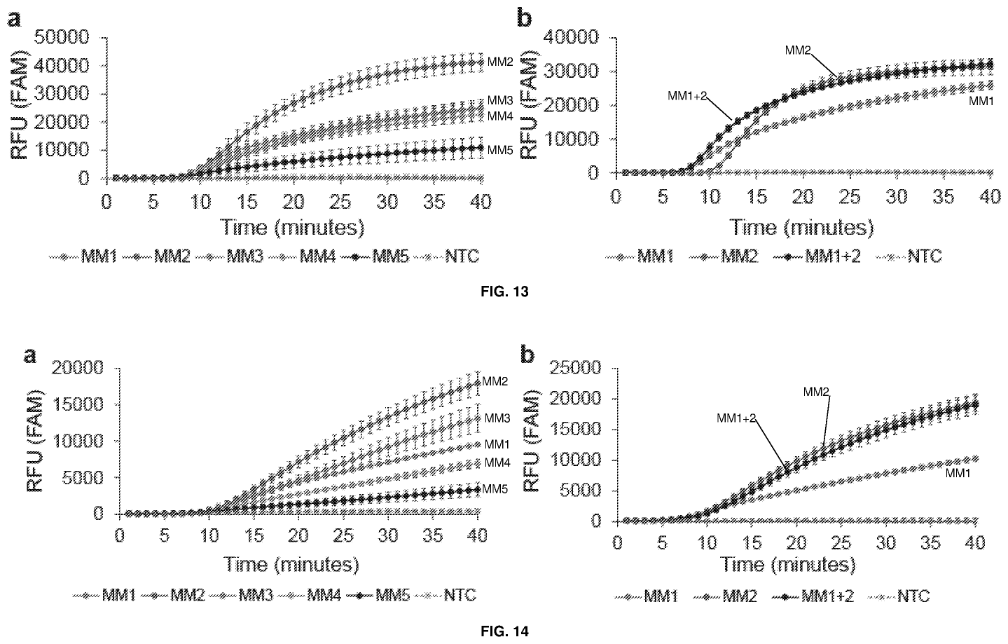

Interestingly, it was observed that a mismatch at the second last position (MM2) yielded the highest fluorescence signal (FIG. 12b and FIG. 13a). This may be because with such a mismatch, the last nucleotide of the probe may also not be able to bind to the template, thereby resulting in a double mismatch at the 3′ end, which might stimulate the proofreading activity of the high fidelity DNA polymerase even more than a single mismatch at the 3′ end. Consistently, when another synthetic template with two mismatches to the probe (MM1+2) was created, it was observed that the fluorescence signal was significantly higher than that of a single mismatch at the 3′ end (MM1) (P<0.01, one-sided Student's t test) but was similar to that of a single mismatch at the second last position (MM2) (FIG. 12c and FIG. 13b). It was further verified that a mismatch at the penultimate position gave the highest fluorescence readout with a different probe that targeted the loop region of the amplicon instead (FIG. 12d-f and FIG. 14). The loop targeting probes and their sequences are listed in Table 5.

TABLE 5
List of probes used (bold and underlined
font indicating the mismatch nucleotide)

| | SEQ ID | |
|---|---|---|
| Name | NO | Sequence |
| Probe | 41 | CAGCTTATTATGTGGGTTATCG |
| MM1 (WT) | 42 | CAGCTTATTATGTGGGTTATCT |
| MM2 | 27 | CAGCTTATTATGTGGGTTATGG |
| MM3 | 43 | CAGCTTATTATGTGGGTTAACG |
| MM4 | 44 | CAGCTTATTATGTGGGTTCTCG |
| MM5 | 45 | CAGCTTATTATGTGGGTAATCG |
| MM1+2 | 46 | CAGCTTATTATGTGGGTTATGT |

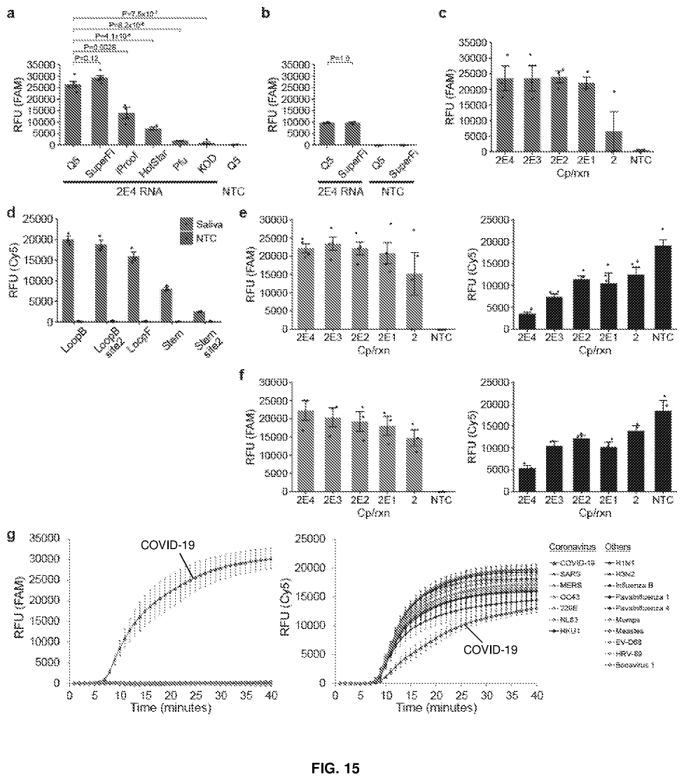

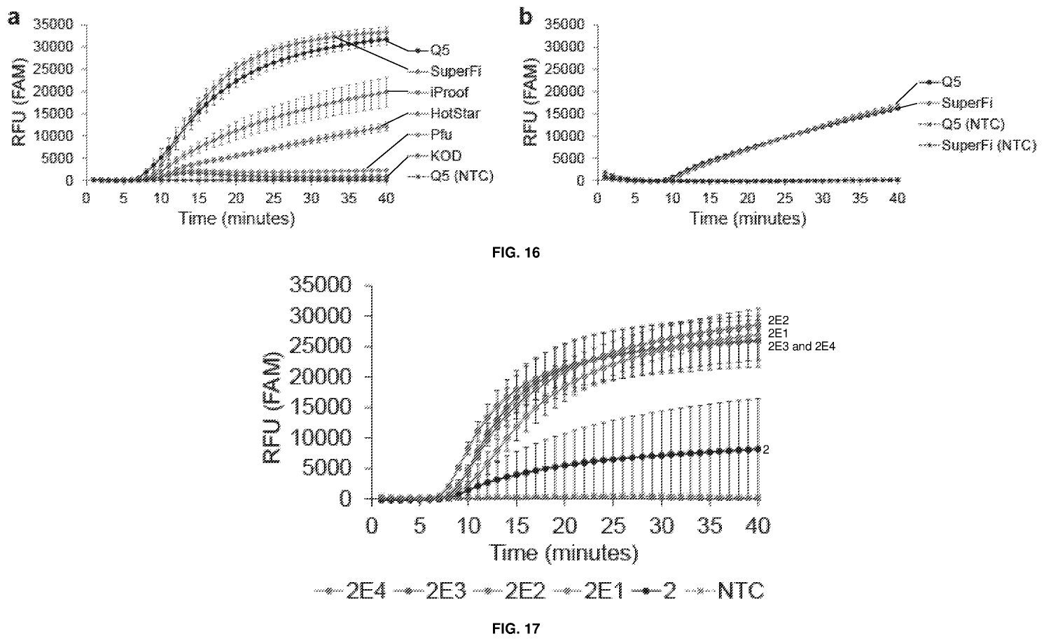

Many different DNA polymerases can potentially be employed in the LANTERN assay. Hence, various commercially available enzymes were evaluated using the double mismatched (MM1+2) probe against the stem region of the S-gene amplicon. First, different high-fidelity DNA polymerases were tested with proofreading capability to cleave the mismatched probe and separate the fluorophore and quencher (FIG. 15a and FIG. 16a). Two of them, namely the Q5 DNA polymerase and the Platinum SuperFi enzyme, clearly outperformed the rest. In particular, the Q5 enzyme, which had been used so far, gave significantly higher fluorescence signals than several other high-fidelity polymerases derived from the thermophilic archaea Pyrococcus (iProof, HotStar, and Pfu) and Thermococcus (KOD) (P<0.01, Student's t test). Second, for the LAMP reaction itself, the Bsm DNA polymerase (derived from Bacillus smithii) was tested with either Q5 or SuperFi (FIG. 15b and FIG. 16b). However, it was found that it gave fluorescence readings that were less than half of those produced by the original Bst DNA polymerase (derived from Bacillus stearothermophilus) (FIG. 15a and FIG. 16a). Hence, these results indicate that the Q5 high-fidelity polymerase and the Bst polymerase are the best combination of enzymes to use in the RT-LAMP assay. Q5 is favoured over the SuperFi enzyme despite both yielding similar fluorescence outputs because the former is much cheaper than the latter, which will lower the cost of any subsequent diagnostic test.

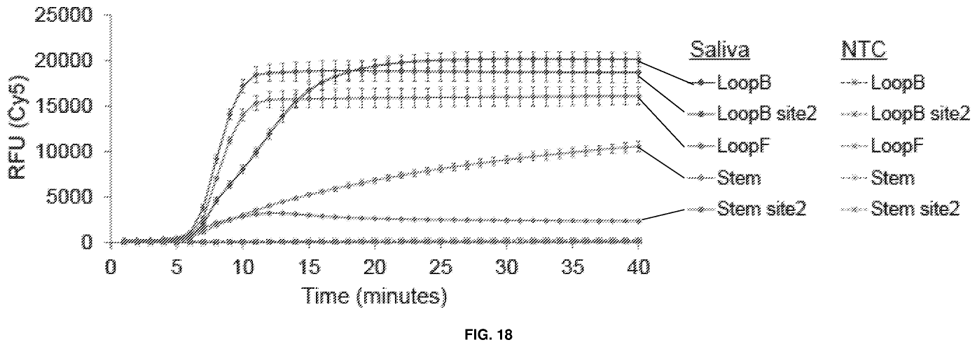

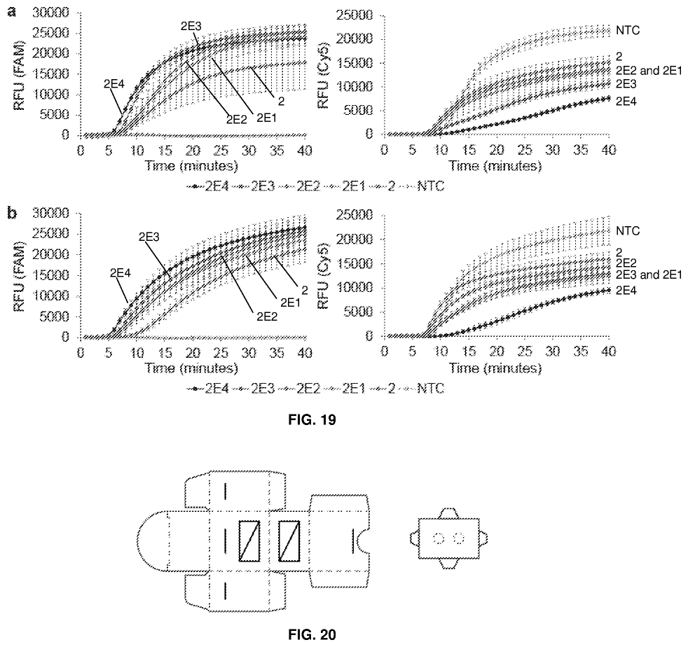

After confirming the best enzymes to use, the sensitivity and specificity of the LANTERN assay was evaluated with double mismatched probes. The analytical LoD of the Sgene probe on the artificial MM1+2 template remained at 20 copies per reaction (FIG. 15c and FIG. 17), but the fluorescence readings were higher than those obtained with a MM1 template especially at lower viral copy numbers (FIG. 2f and FIG. 6a). Hence, to enhance the signal for the internal control, several ACTB probes with two 3′ end mismatches were also evaluated using healthy donor saliva (FIG. 15d and FIG. 18). The probe that hybridized to the original loopB region gave the highest fluorescence readings. Subsequently, the LANTERN assay was assessed in a multiplexed format. A new viral probe that contained two 3′ end mismatches against the wild type S-gene together with the double mismatched loopB targeting human ACTB probe in a one-pot reaction. The viral probe was conjugated with FAM, while the human probe was conjugated with Cy5. In six of the seven replicates, 2 copies of in vitro transcribed SARS-CoV-2 RNA were able to be detected that had been spiked into total human RNA (FIG. 15e-f and FIG. 19). Furthermore, the assay was evaluated against a set of coronaviruses and other respiratory viruses, including influenza viruses, paramyxoviruses, and enteroviruses, by spiking each of the viral RNAs individually into total human RNA. Over the course of 40 min, fluorescence was detected in the Cy5 channel for all viruses, but in the FAM channel for SARS-CoV-2 only (FIG. 15g). Collectively, the results indicate that the LANTERN assay for COVID-19 is highly sensitive and specific.

### Example 4: Implementation of the LANTERN Assay Using Low-Cost Components

To promote frequent decentralized testing, COVID-19 rapid assays should be as affordable and straightforward-to-use as possible. Hence, the LANTERN assay was implemented in a simple, low-cost format without utilizing a real-time PCR machine. An immediate question is how a user can obtain the test results without any sophisticated laboratory equipment. To address the issue, it was decided to construct a simple lightbox out of common cardboard material. A drawing in a manner similar to that of an origami project (FIG. 20). After cutting the shape out of a piece of cardboard, it can be folded it into a box, pasted colour filter papers over the windows, and inserted the tube holder. When illuminated with light from a mobile phone or from a LED, the fluorescence signals from sample tubes were readily visualized. Importantly, the cardboard, filters, and LED can be easily purchased from art and craft stores, electronics hobby stores, or online shopping platforms.

Subsequently, the multiplex LANTERN assay was demonstrated with a heat block and the homemade lightboxes. To this end, the viral S-gene and human ACTB primers and probes were pooled together into a one-pot reaction. The viral probe was conjugated with FAM, while the human probe was conjugated with JUN. Variable copies of synthetic SARS-CoV-2 RNA were spiked into total RNA from the human PC9 cell line as before (FIG. 15f and FIG. 19b), but this time, all the sample tubes were incubated on a 65° C. heat block for 30 min before visualizing the fluorescence in the lightboxes. Encouragingly, 20 copies of viral RNA were able to be detected in all the replicates and 2 copies of the RNA in 2 out of the 3 replicates. Hence, the analytical LoD remained similar regardless of whether the experiments were performed using an expensive real-time PCR instrument or cheap hardware components. Moreover, all the sample tubes exhibited fluorescence in the lightbox with a 650 nm long-pass filter, which corresponded to the JUN conjugated probe for the human ACTB internal control. Collectively, the results show that users can perform the multiplex LANTERN assay using low-cost components and without needing any scientific expertise, since test outcomes can be readily obtained by naked eye.

### Example 5: Direct Testing of Swab and Saliva Samples without RNA Extraction

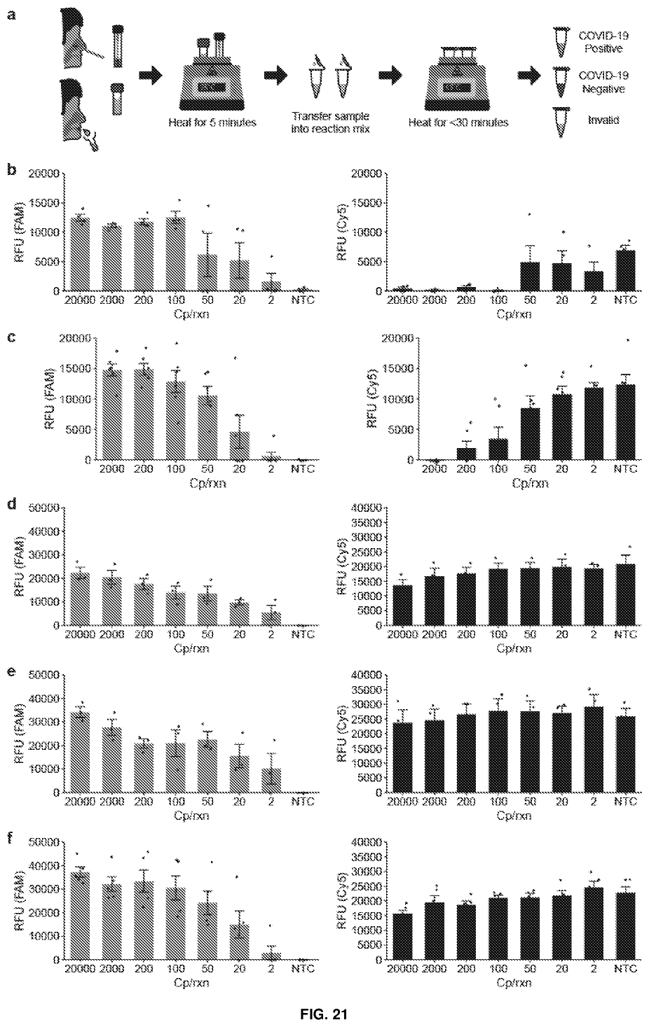

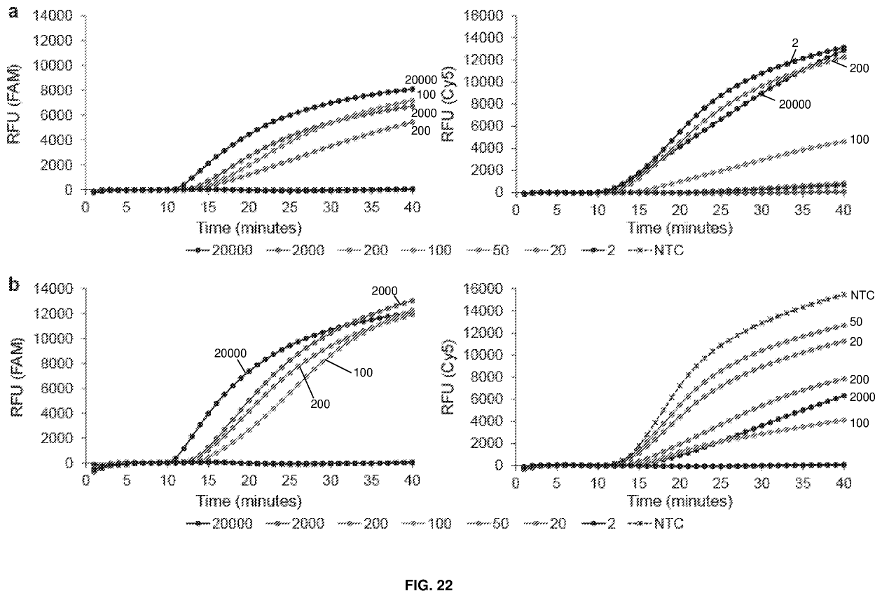

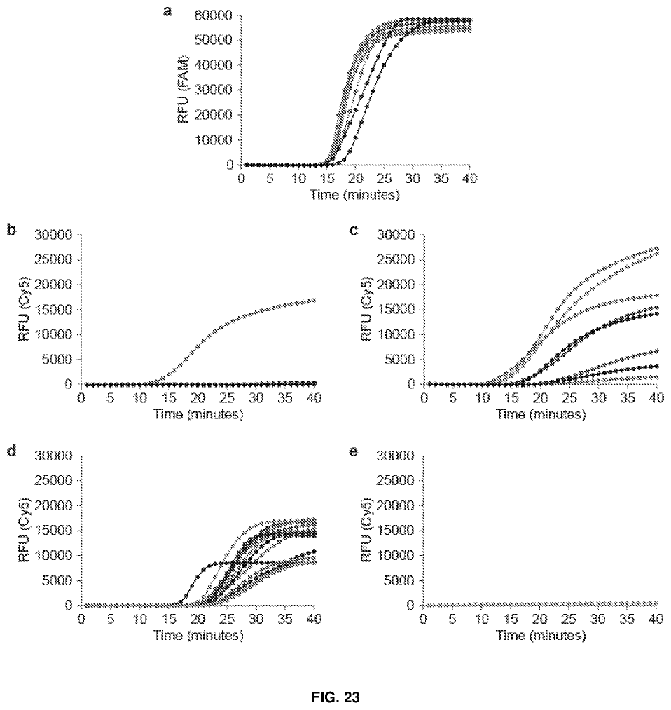

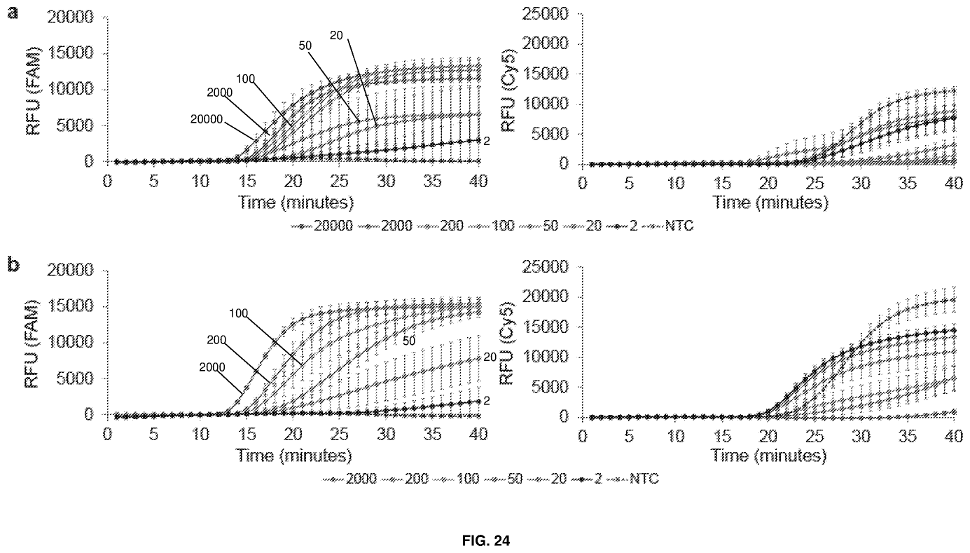

A key consideration of rapid point-of-care or point-of-need diagnostic tests is whether they can accommodate clinical samples directly without an extra RNA purification step, which typically takes at least 15 min and increases complexity of the workflow. Hence, the use of the LANTERN assay was investigated on clinical samples directly without any RNA extraction (FIG. 21a). As simulation, different amounts of commercially available heat-inactivated SARS-CoV-2 virus produced by Vero E6 cells were first spiked into clinically negative Universal Transport Media (UTM) for testing. To inhibit any RNases present and further lyse any remaining intact viral particles, the contrived specimens were then treated with proteinase K and 95° C. heat before loading them into reaction tubes, a procedure previously validated.¹³ Unexpectedly, however, it was not possible to consistently detect the ACTB internal control (FIG. 22). This might be because the UTM samples contain some substance that can inhibit Q5 in the reaction, especially since the DNA polymerase is not known to be a very inhibitor tolerant enzyme. Hence, to boost detection, the amount of Q5 polymerase was increased to 2 U in a 25 μL reaction volume. Encouragingly, the human internal control could now be reliably detected in all the replicates when there was no viral RNA present (FIG. 23). Nevertheless, it was observed that amplification of ACTB still remained challenging in the presence of the virus, especially at higher viral loads (FIG. 21b and FIG. 24). Moreover, for these contrived UTM specimens with spiked-in SARS-CoV-2, the assay sensitivity was only 100 copies per reaction, which was appreciably lower than that for synthetic RNA samples (FIG. 15e,f and FIG. 19). Hence, 50 mM EDTA was added into the lysis solution, which chelates divalent cations like Mg2+ and helps to protect RNA from degradation. The additional EDTA improved the analytical LoD for contrived swab samples to 50 copies per reaction and further enhanced the amplification of human ACTB (FIG. 21c and FIG. 24b).

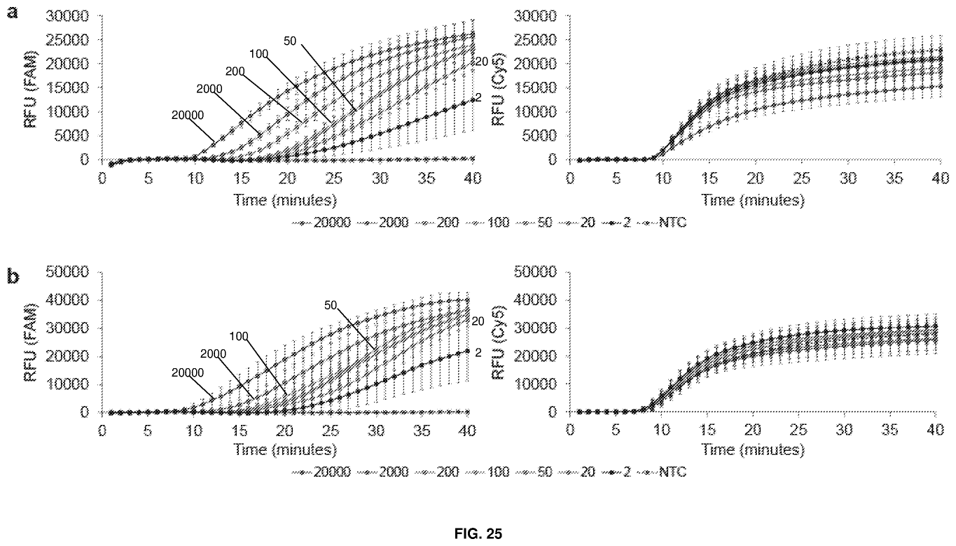

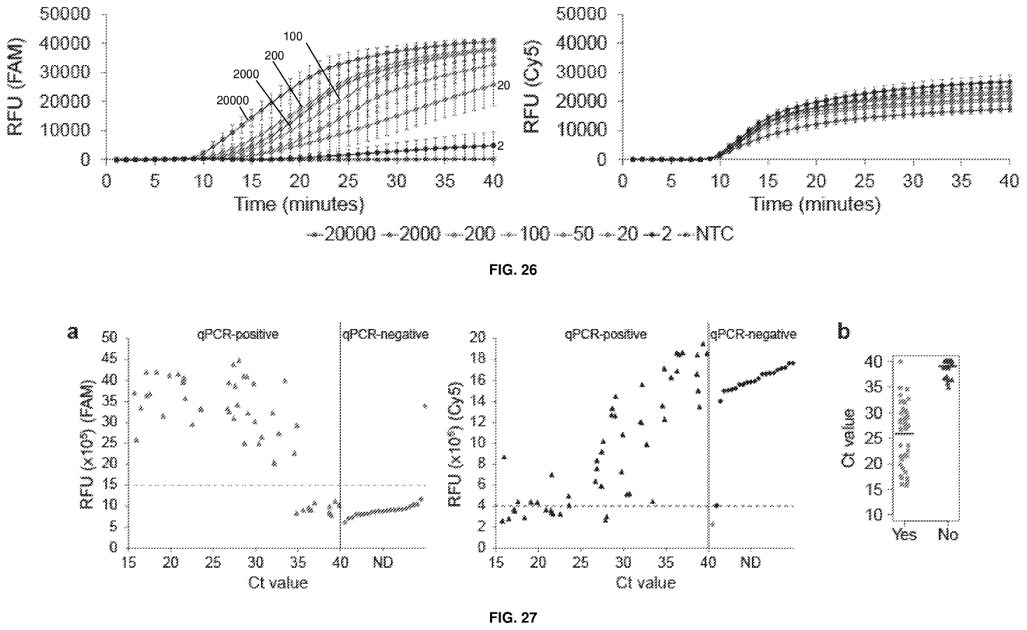

Saliva is increasingly being utilized as an alternative diagnostic sample as its collection method is simpler and less invasive than NP swabs. Hence, to evaluate the applicability of the assay on saliva samples, variable amounts of SARS-CoV-2 virus produced by Vero E6 cells were spiked into donor saliva and treated each sample with proteinase K and 95° C. heat before adding it into the RT-LAMP reaction mix containing the LANTERN probes. 0.5 U of Q5 polymerase was used without any EDTA. It was found that the analytical LoD was 20 copies per reaction, regardless of whether the reaction volume was 25 μL (FIG. 21d and FIG. 25a) or 50 μL (FIG. 21e and FIG. 25b), although a larger volume yielded an overall higher fluorescence signal. Notably, the human internal control successfully amplified in every replicate for all the virus concentrations tested. To confirm the results, the contrived specimens were further tested with a commercially available ZeroPrep Saliva Collection Kit, which contained proteinase K in the buffer and also required a 95° C. heating step (FIG. 21f and FIG. 26). With the kit, 50 and 20 copies of the virus could be detected in 100% and 80% of the replicates, respectively. Moreover, strong fluorescence signals were again detected for ACTB in the Cy5 channel regardless of the amount of virus present. Taken together, the results indicate that the multiplexed LANTERN diagnostic test can be readily applied to saliva samples without any modification to the assay components and may further be applied to swab samples with extra Q5 enzyme and EDTA in the reaction and lysis buffers, respectively.

### Example 6: Evaluation of LANTERN Assay with Clinical RNA Samples

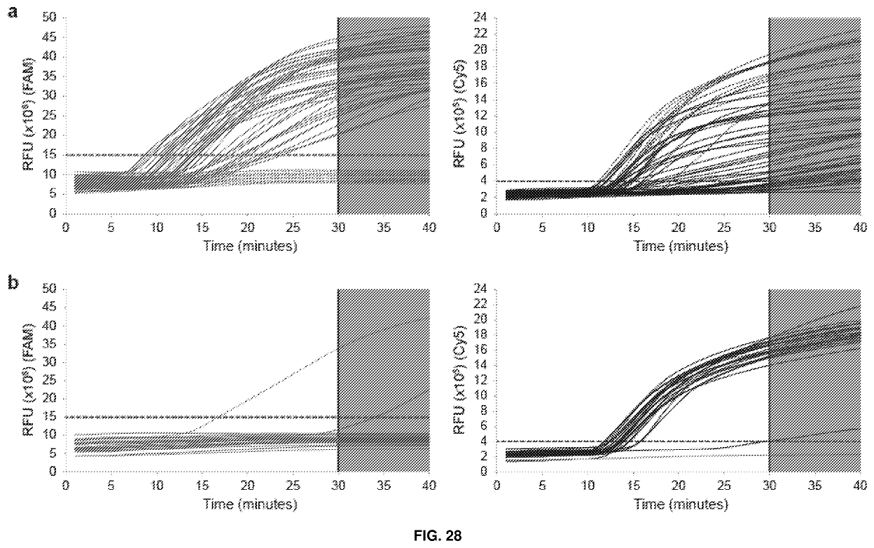

To benchmark the fluorescent RT-LAMP assay, it was subjected to independent clinical evaluation with residual RNA samples isolated from patient NP swabs, which had previously been analyzed by RT-qPCR in Thailand. These samples came from 52 individuals diagnosed with COVID-19 and 22 uninfected persons. Samples that exhibited a wide range of Ct values (from 15 to 40) were selected to obtain a more accurate picture of the assay sensitivity. Fluorescence was monitored in a real-time PCR machine (FIG. 27a and FIG. 28). Among the COVID-19 negative samples, one returned an invalid result as the ACTB internal control did not amplify, likely due to inadequate material leftover. In the remaining 21 samples, the LANTERN diagnostic test also returned a negative result in 20 of them, giving the assay a specificity of 95.2%. Nevertheless, when the single false positive sample was re-evaluated, the S-gene and the ACTB control was unable to amplify again in the RT-LAMP assay, suggesting that the sample had already degraded and that the earlier false positive result may be due to accidental cross-contamination. For the virus infected samples, a clear separation was observed in fluorescence signals between those that yielded positive results and those that did not. Overall, the LANTERN test returned an unambiguous positive outcome for clinical samples that had a Ct value of 34.6 or lower in RT-qPCR analysis (FIG. 27b). This translated to a clinical LoD of around 8 copies per reaction or 0.32 copies per microliter. It was also noted that for samples with high viral loads (Ct less than 25), the human internal control was less readily detected, which was unsurprising since the ACTB LAMP primers were loaded at a 3-fold lower concentration than the Sgene primers. Collectively, these results demonstrate that the LANTERN assay using the newly designed probes can be successfully applied on clinical RNA samples to rapidly diagnose COVID-19 with high sensitivity and specificity.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other embodiments are within the following claims.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The methods, kits and uses described herein are presently representative of preferred embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The content of all documents and patent documents cited herein is incorporated by reference in their entirety.

### REFERENCES

(1) Dao Thi, V. L.; et al. A colorimetric RT-LAMP assay and LAMP-sequencing for detecting SARS-CoV-2 RNA in clinical samples. Sci. Transl. Med. 2020,
(2) Lamb, L. E.; et al. Rapid detection of novel coronavirus/Severe Acute Respiratory Syndrome Coronavirus 2 (SARS-CoV-2) by reverse transcription loop-mediated isothermal amplification. PLOS One 2020, 15 (6), e0234682.
(3) Zhang, Y.; et al. A. Enhancing colorimetric loop-mediated isothermal amplification speed and sensitivity with guanidine chloride. Biotechniques 2020, 69 (3), 178-185.
(4) Mautner, L.; et al. Rapid point of-care detection of SARS-CoV-2 using reverse transcription loop mediated isothermal amplification (RT-LAMP). Virol J. 2020, 17 (1), 160.
(5) Buck, M. D.; et al. SARS-CoV-2 detection by a clinical diagnostic RT-LAMP assay. Wellcome Open Res. 2021, 6, 9.
(6) Mayboroda, O.; et al. Multiplexed isothermal nucleic acid amplification. Anal. Biochem. 2018, 545, 20-30. Chou, P. H.; et al. Real-time target-specific detection of loop-mediated isothermal amplification for white (7) spot syndrome virus using fluorescence energy transfer-based probes. J. Virol Methods 2011, 173 (1), 67-74.
(8) Ding, S.; et al. Sequence-specific and multiplex detection of COVID-19 virus (SARS-CoV-2) using proofreading enzyme-mediated probe cleavage coupled with isothermal amplification. Biosens Bioelectron 2021, 178, 113041.
(9) Koksaldi, I. C.; et al. SARS-CoV-2 Detection with De Novo-Designed Synthetic Riboregulators. Anal. Chem. 2021, 93 (28), 9719-9727.
(10) Park, S.; Lee, J. W. Detection of Coronaviruses Using RNA Toehold Switch Sensors. Int. J. Mol. Sci. 2021, 22 (4), 1772.
(11) Sherrill-Mix, S.; et al. Detection of SARS-CoV-2 RNA using RT-LAMP and molecular beacons. Genome Biol. 2021, 22 (1), 169.
(12) Bhadra, S.; et al. High-Surety Isothermal Amplification and Detection of SARSCoV-2. mSphere 2021
(13) Ooi, K. H.; et al. An engineered CRISPR-Cas12a variant and DNA-RNA hybrid guides enable robust and (13) rapid COVID-19 testing. Nat. Commun. 2021, 12 (1), 1739.
(14) Farfour, E.; et al. The Allplex 2019-nCOV (Seegene) assay: which performances are for SARS-CoV-2 infection diagnosis? Eur. J. Clin Microbiol Infect Dis 2020, 39 (10), 1997-2000.

1. A method for determining the presence or quantity of a target nucleic acid molecule in a sample using isothermal amplification, the method comprising:
(a) combining an isothermal amplification reaction mixture, a DNA polymerase with 3′-5′ exonuclease activity, and a detection probe with the sample (suspected of containing the target nucleic acid molecule),
wherein the isothermal amplification reaction mixture comprises a primer set of at least two primers, wherein each primer recognises a distinct primer binding site within the target nucleic acid molecule,
wherein the detection probe is a single-stranded probe that recognises a probe binding site within target amplicons, said probe binding site being different from and non-overlapping with any one of the primer binding sites, and
wherein the detection probe comprises at least one 3′ end nucleotide mismatch and a quencher-fluorophore pair at opposite ends of the probe at a distance that allow the quencher to quench the fluorophore signal, wherein either the fluorophore or the quencher are attached to the 3′ end of the probe downstream of or at the site of the mismatch,
wherein the detection probe can hybridize to said target amplicons under isothermal amplification assay conditions except for the 3′ end nucleotide mismatch and form a double-stranded probe:target complex,
(b) amplifying the target nucleic acid molecule under isothermal amplification assay conditions that allow:
i. generation of the target amplicons;
ii. hybridization of the detection probe to the target amplicons to form the probe:target complex; and
iii. cleavage of the detection probe at the 3′ end nucleotide mismatch by the DNA polymerase with 3′-5′ exonuclease activity to release a 3′-terminal probe fragment comprising the quencher or fluorophore; and
(c) detecting and optionally quantifying the released probe fragments to determine the presence and optionally amount of the target nucleic acid molecule in the sample.

2. The method of claim 1, wherein the DNA polymerase with 3′-5′ exonuclease activity is a high-fidelity DNA polymerase.

3. The method of claim 1, wherein the isothermal amplification is loop-mediated isothermal amplification (LAMP), and the primer set comprises at least 4 or 6 primers comprising two inner primers (FIP and BIP) and two outer primers (F3 and B3), and optionally two loop primers (LF and LB).

4. The method of claim 3, wherein the probe binding site lies between the binding sites of the inner primers.

5. The method of claim 3, wherein the primer set further comprises two swarm primers.

6. The method of claim 1, wherein the at least one 3′ end nucleotide mismatch comprises a single 3′ end nucleotide mismatch.

7. The method of claim 6, wherein the single 3′ end nucleotide mismatch is positioned at the last or second to last nucleotide relative to the 3′ end of the detection probe.

8. The method of claim 1, wherein the at least one 3′ end nucleotide mismatch comprises two 3′ end nucleotide mismatches.

9. The method of claim 8, wherein the two 3′ end nucleotide mismatches are the last two nucleotides relative to the 3′ end of the detection probe.

10. The method of claim 1, wherein the detection probe is 17-30 nucleotide bases in length.

11. The method of claim 1, wherein the quencher is attached to the 5′ end of the detection probe and the fluorophore is attached to the 3′ end of the detection.

12. The method of claim 1, wherein the quencher is a double quencher.

13. The method of claim 1, wherein the detection method in step (c) is lateral flow detection or fluorescence detection.

14. The method of claim 1, wherein the method is a multiplexing method and is for determining the presence, absence and optionally amount of two or more target nucleic acid molecules in the sample, wherein the method uses one or more primer sets and/or one or more detection probes for each target nucleic acid molecule or for multiple related target nucleic acid molecules.

15. The method of claim 1, wherein the target nucleic acid molecule is a nucleic acid of a pathogen, optionally a human pathogen, preferably a bacterial, fungal, parasite or viral nucleic acid molecule or a cDNA reverse transcript of a bacterial, fungal, parasite or viral RNA.

16. The method of claim 15, wherein the target nucleic acid molecule is a nucleic acid of a coronavirus, influenza virus, paramyxovirus or enterovirus.

17. The method of claim 16, wherein the target nucleic acid molecule is a nucleic acid of SARS-CoV-2 virus.

18. The method of claim 1, wherein the sample has not been subjected to any nucleic acid purification or extraction step prior to step (a) of the method.

19. The method of claim 1, wherein step (a) further comprises pyrophosphatase.

20. (canceled)

21. A kit for determining the presence or quantity of a target nucleic acid molecule in a sample using isothermal amplification, the kit comprising:
an isothermal amplification reaction mixture;
a DNA polymerase with 3′-5′ exonuclease activity; and
a detection probe,
wherein the isothermal amplification reaction mixture comprises a primer set of at least two primers, wherein each primer recognizes a distinct primer binding site within the target nucleic acid molecule,
wherein the detection probe is a single-stranded probe that recognizes a probe binding site within target amplicons, said probe binding site being different from and non-overlapping with any one of the primer binding sites,
wherein the detection probe comprises at least one 3′ end nucleotide mismatch and a quencher-fluorophore pair at opposite ends of the probe at a distance that allow the quencher to quench the fluorophore signal, wherein either the fluorophore or the quencher are attached to the 3′ end of the probe downstream of or at the site of the mismatch,
wherein the detection probe can hybridize to said target amplicons under isothermal amplification assay conditions except for the 3′ end nucleotide mismatch and form a double-stranded probe:target complex.
